(19) United States
(12) Patent Application Publication
(10) Pub. No.: US 2005/0112130 A1
(43) Pub. Date: May 26, 2005

(54) Enhanced B cell cytotoxicity of CDIM binding antibody

(76) Inventors: Neelima M. Bhat, Los Altos, CA (US); Marcia M. Bieber, Los Altos, CA (US); Nelson N.H. Teng, Hillsborough, CA (US); Martin E. Sanders, Hillsborough, CA (US)

Correspondence Address: CYNTHIA RUTH MOORE, 794 LOS ROBLES AVENUE, PALO ALTO, CA, 94306, US

(21) Appl. No.: 10/982,698

(22) Filed: Nov. 5, 2004

Related U.S. Application Data
(60) Provisional application No. 60/517,775, filed on Nov. 5, 2003.

(51) Int. Cl. A61K 39/395; A61K 39/42

(52) U.S. Cl. 424/155.1; 424/159.1; 514/2

(57) ABSTRACT

Formulations and methods of treating human patients suffering from a condition characterized by lymphoid cancer, autoimmune disease or B cell hyperproliferation are disclosed, the treatment comprising administering (1) a cytotoxic amount of an antibody having specific binding for CDIM epitopes on a B cell, and (2) a cytotoxic agent, including a chemotherapeutic agent, radioactive isotope, cytotoxic antibody, immunoconjugate, ligand conjugate, immunosuppressant, cell growth regulator and/or inhibitor, toxin, or mixtures thereof, including agents that disrupt the cytoskeleton of B cells, particularly vinca alkaloids or colchicine.

This application claims benefit of U.S. Provisional Patent Application No. 60/517,775, filed Nov. 5, 2003, which is incorporated herein by reference.

### FIELD OF THE INVENTION

This invention relates generally to compositions and methods for treating cancer and hyperproliferative diseases and the like.

### BACKGROUND OF THE INVENTION

Acute lymphoblastic leukemia (ALL) is the most common malignancy of childhood. Approximately 80% of childhood ALL is of B-cell lineage. Although with current therapy nearly 80% of children with ALL will be cured, for the remaining group of patients the need for new and different treatment strategies continues to be a therapeutic challenge. For children who suffer a bone marrow relapse of their leukemia post-allogeneic bone marrow transplantation (BMT), the probability of cure is slim. Similarly, children who have not received a BMT due to lack of a suitable donor and who have had at least two relapses of their disease, are unlikely to be cured with traditional chemotherapy. Under these circumstances, it may be difficult to achieve a complete remission with reinduction chemotherapy, both due to the refractory nature of the leukemia as well as the potentially fragile nature of the patient, who has been heavily pretreated. Therefore, the need to develop novel agents that either alone, or in combination with chemotherapy, are active against ALL remains a goal of modern leukemia therapy. Agents which demonstrate specificity for leukemic blasts but which do not share a similar toxicity profile with chemotherapy drugs would be particularly advantageous for designing new strategies of anti-leukemia therapy. In addition, it is desirable to discover agents and methods that could increase the efficacy of existing chemotherapeutic or biologic agents in the treatment of other B cell cancers, including chronic lymphocytic leukemia (CLL) and lymphomas of B cell lineage, as well as autoimmune disease mediated by B cells.

MAb 216, described in U.S. Pat. Nos. 5,593,676 and 5,417,972, and EP 0 712 307B1, all commonly assigned, describes the use of an antibody that binds a CDIM epitope for killing B cells. Variable amounts of B cells can be killed using this antibody, and enhanced efficacy is desired for treating diseases characterized by a hyperproliferation of B cells such as lymphoid cancers.

### SUMMARY OF THE INVENTION

Accordingly, it is a primary object of the invention to address the aforementioned need in the art by providing novel methods and pharmaceutical formulations for combating lymphoid cancer and other disorders characterized by hyperproliferation of B cells.

Accordingly, in one embodiment, a method is provided for treating a human or other mammalian species expressing the CDIM antigen restricted to cells of B cell lineage, wherein the mammal is suffering from a condition characterized by a hyperproliferation of B cells. The method comprises contacting said B cells with (1) a cytotoxic amount of an antibody having specific binding for CDIM epitopes on a B cell, and (2) a cytotoxic agent. In a preferred aspect, the condition characterized by a hyperproliferation of B cells is lymphoid cancer, viral infection, immunodeficiency, or autoimmune disease. Representative viral infections include human immunodeficiency virus or mononucleosis. Representative immune deficiencies include post-transplant lymphoproliferative disease or immunodeficiency syndrome, and can be found in patients receiving anticancer therapies or other immunosuppressive therapies. Representative autoimmune diseases include systemic lupus erythematosis, rheumatoid arthritis, autoimmune lymphoproliferative disease, multiple sclerosis, psoriasis, and myasthenia gravis, but can also include Hashimoto's thyroiditis, lupus nephritis, dermatomyositis, Sjogren's syndrome, Sydenham's chorea, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, Crohn's disease, Alzheimer's disease, sarcoidosis, ulcerative colitis, erythema multiforme, IgA nephropathy, polyarteritis nodosa, ankylosing spondylitis, Goodpasture's syndrome, thromboangitis ubiterans, primary biliary cirrhosis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, fibrosing alveolitis, Class III autoimmune diseases such as immune-mediated thrombocytopenias, such as acute idiopathic thrombocytopenic purpura and chronic idiopathic thrombocytopenic purpura, and the like.

The cytotoxic agent can be a chemotherapeutic agent, a radioactive isotope, a cytotoxic antibody, an immunoconjugate, a ligand conjugate, an immunosuppressant, a cell growth regulator and/or inhibitor, a toxin, or mixtures thereof. The chemotherapeutic agent can be an agent that disrupts the cytoskeleton of the B cell. In additional embodiments, the chemotherapeutic agent can be asparaginase, epipodophyllotoxin, camptothecin, antibiotic, platinum coordination complex, alkylating agent, folic acid analog, pyrimidine analog, purine analog or topoisomerase inhibitor, or mixtures thereof.

Preferably, the agent that disrupts the cytoskeleton of the B cell is an agent that interferes with the polymerization or depolymerization of microtubules, such as a taxane, vinca alkaloid and colchicine, or mixtures thereof. Vinca alkaloids include, for example, vinblastine, vincristine, vindesine, or vinorelbine, or mixtures thereof. Taxanes include paclitaxel, and docetaxel, and mixtures thereof. In another embodiment, the agent that disrupts the cytoskeleton of the B cell is an anti-actin agent, such as jasplakinolide and cytochalasin.

Topoisomerase inhibitors include epipodophyllotoxins, such as etoposide or teniposide. Pyrimidine analogs include, without limitation, capecitabine, 5-fluoruracil, 5-fluorodeoxyuridine, 5-fluorodeoxyuridine monophosphate, cytosine arabinoside, 5-azacytidine, 2′, 2′-difluorodeoxycytidine. Purine analogs include mercaptopurine, azathioprine, thioguanine, pentostatin, erythrohydroxynonyladenine, cladribine, vidarabine, fludarabine phosphate, for example. Folic acid analogs include methotrexate, raltitrexed, lometrexol, permefrexed, edatrexate, pemetrexed. Camptothecins include irinotocan, topotecan, camptothecan. Antibiotics include dactinomycin, daunorubicin, doxorubicin, idarubicin, epirubicin, valrubucin, mitoxanthrone, bleomycin, and mitomycin, without limitation. Platinum coordination complexes include cisplatin, carboplatin, and oxaliplatin, for example. Alkylating agents include, for example, mechlorethamine, cyclophosphamide, ifosfamide, melphalan, dacarbazine, temozolomide, thiotepa, hexamethylmelamine, streptozocin, carmustine, busulfan, altretamine and chlorambucil.

The cytotoxic agent can be administered simultaneously with, before or after administration of the antibody having specific binding for CDIM epitopes on a B cell. For example, by administering a cytotoxic amount of an antibody having specific binding for CDIM epitopes on a B cell to a patient suffering from lymphoid cancer prior to treatment with conventional chemo- or immunotherapy, a method is provided for reducing tumor load in the patient. For example, when the patient becomes refractory to reinduction therapy, administering the antibody having specific binding for CDIM epitopes on a B cell allows the patient to undergo subsequent reinduction therapy. The method can further comprise treating the patient with a cytotoxic agent.

In another embodiment, there is provided a method for purging the bone marrow of a patient suffering from lymphoid cancer of malignant B cells prior to reimplantation of the bone marrow in the patient after myeloablative therapy. The method comprises treating the bone marrow ex vivo with a cytotoxic amount of an antibody having specific binding for CDIM epitopes on a B cell. The method can further comprise treating the bone marrow cells ex vivo with a cytotoxic agent.

The cytotoxic amount of an antibody having specific binding for CDIM epitopes on a B cell induces a cell membrane wound which results in permeabilization of the B cell to chemotherapeutic agents, as well as other cytotoxic agents which may have enhanced efficacy once access to the B cell cytosol is facilitated by the cell membrane wound. Accordingly, by administering a cytotoxic amount of an antibody having specific binding for CDIM epitopes on a B cell prior to, during or even after treatment with conventional chemotherapy, methods are provided for augmenting the cytotoxicity of chemotherapeutic agents, thereby enhancing the efficacy of chemotherapy. Further, this enhancement in efficacy of chemotherapy may allow for the treatment of patients using lower concentrations of chemotherapeutic agents, thereby providing an efficacious treatment with potentially fewer side effects and adverse events.

Similarly, by administering a cytotoxic amount of an antibody having specific binding for CDIM epitopes on a B cell prior to, during or even after treatment with conventional immunotherapy, methods are provided for augmenting the cytotoxicity of an anti-B cell antibody utilized during immunotherapy. In addition, conventional anti-B cell immunotherapy may lack efficacy under conditions of high tumor load or immunodeficiency, such as when complement stores become depleted, and anti-B cell immunotherapy is rendered inefficacious. The combination with an antibody having specific binding for CDIM epitopes on a B cell overcomes this lack of efficacy of conventional anti-B cell immunotherapy, e.g., where there is a complement deficiency. Therefore it can be most advantageous to administer cytotoxic agents prior to or during administration of the antibody having specific binding for CDIM epitopes on a B cell, as this antibody induces cell wounding, enhancing both the efficacy of the antibody and the cytotoxic agent.

The antibody having specific binding for CDIM epitopes on a B cell can be a natural antibody, monoclonal antibody, a polyclonal antibody, a chimeric antibody, a human antibody, a humanized antibody, a single-chain Fv antibody, an antibody fragment. (e.g., Fab), a pegylated antibody, tetravalent antibody, a diabody, or a minibody, or the like, so long as cell membrane permeabilization and/or cytotoxicity is provided by the antibody. The antibody having specific binding for CDIM epitopes on a B cell can also prepared as a fusion protein comprising a heterologous polypeptide to form an immunoconjugate comprising a cytotoxic agent, or it can be covalently or noncovalently modified to comprise a cytotoxic agent such as a radioactive isotope or toxin. Preferably, when the antibody having specific binding for CDIM epitopes on a B cell is conjugated to, labeled with, or fused to a cytotoxic agent, the full size antibody is utilized so as to take advantage of the cell wounding cytotoxicity provided by the antibody as well as the additional cytotoxicity provided by the cytotoxic agent.

In particular aspects, the antibody having specific binding for CDIM epitopes on a B cell is a VH4-34 encoded antibody. Preferred members of this antibody family include mAb 216, RT-2B, FS 12, A6(H4C5), Cal-4G, S20A2, FS 3, Gee, HT, Z2D2, Y2K. Preferred antibodies having specific binding for CDIM epitopes on a B cell comprise a CDR sequence having a net positive charge.

In certain embodiments, the cytotoxic agent is a radioactive isotope, for example, ¹³¹I, ¹²⁵I, ¹²³I, ⁹⁰Y, ¹¹¹In, ¹⁰⁵Rh, ¹⁵³Sm, ¹⁶⁶Ho, ¹⁷⁷Lu, and ¹⁸⁸Re and ¹⁸⁶Re, ³²p, ⁵⁷Co, ⁶⁴Cu, ⁶⁷Cu, ⁷⁷Ga, ⁸¹Rb, ⁸¹Kr, ⁸⁷Sr, ¹¹³In, ¹²⁷Cs, ¹²⁹Cs, ¹³²I, ¹⁹⁷Hg, ²¹³Pb, ²¹⁶Bi, ¹¹⁷Lu, ²¹²Pb, ²¹²Bi, ⁴⁷Sc, ¹⁰⁵Rh, ¹⁰⁹Pd, ¹⁹⁹Au, ²²⁵Ac, ²¹¹At, and ²¹³Bi. Of these radioactive isotopes, ¹³¹I, ¹²⁵I, ⁹⁰Y, ¹¹¹In, and ¹⁸⁶Re are most preferred. The radioactive isotope can comprise a part of an immunoconjugate or ligand conjugate. In certain other embodiments, the radioactive isotope is covalently attached to the antibody having specific binding for CDIM epitopes on a B cell, or to the cytotoxic antibody having specific binding for a cell surface receptor on a B cell.

In particular embodiments, the antibody having specific binding for CDIM epitopes on a B cell is used in combination with an additional cytotoxic antibody having specific binding for cell surface molecules on a B cell. The cytotoxic antibody can have specific binding for any cell surface molecule on a B cell. Cell surface molecules include receptors, immunoglobulins, cytokines, glycoproteins, etc. For example, the cytotoxic antibody can exhibit specific binding for CD11a, CD19, CD20, CD21, CD22, CD25, CD34, CD37, CD38, CD40, CD45, CD52, CD80, CD 86, IL-4R, IL-6R, IL-8R, IL-13, IL-13R, α-4/β-1 integrin (VLA4), BLYS receptor, cell surface idiotypic Ig, tumor necrosis factor (TNF),or mixtures thereof, without limitation. For example, the cytotoxic antibody having specific binding for CD11a can be, for example, efalizumab (RAPTIVA). The cytotoxic antibody having specific binding for CD20 can be rituximab (RITUXAN). The cytotoxic antibody having specific binding for CD22 can be, for example, epratuzumab. The cytotoxic antibody having specific binding for CD25 can be, for example, daclizumab (ZENAPAX) or basiliximab (SIMULECT). Antibodies to CD52 include, e.g., CAMPATH. Antibodies to α-4/β-1 integrin (VLA4) include, e.g., natalizumab. Antibodies to TNF include, for example, infliximab (REMICADE).

Thus in preferred embodiments, the antibody having specific binding for CDIM epitopes on a B cell can be used in a combined immunotherapy regimen with RITUXAN, ZENAPAX, REMICADE or RAPTIVA, for example, or in combinations thereof. The cytotoxic antibody can also be used as an immunoconjugate comprising a radioactive isotope or toxin, for example. Further, in additional embodiments, a combined therapy can be used comprising the antibody having specific binding for CDIM epitopes on a B cell, an additional cytotoxic antibody having specific binding for cell surface molecules on a B cell, and one or more chemotherapeutic agents. For example, mAb216 could be used in combination with an anti-CD20 antibody such as rituximab, tosutimab, or ibritumomab, or in combination with an anti-CD52 antibody such as CAMPATH, or in combination with an anti-CD22 antibody, such as epratuxumab, and so forth. The combination therapy can further include chemotherapy, such as an agent that disrupts the cytoskeleton of the cell, e.g., vincristine, in a combined chemotherapy and immunotherapy regimen.

In additional embodiments, the cytotoxic agent can be a ligand conjugate, which includes any B cell receptor ligand which binds to a cell surface receptor on a B cell. Such ligands include, without limitation, IL-2, IL-4, IL-6, IL-13, IL-15, BLYS, or TNF, or the like. Ligand conjugates, like immunoconjugates, include fusion proteins or covalently or noncovalently bound toxins, radioactive isotopes, or other toxic agent. Thus, in this embodiment, the antibody having specific binding for CDIM epitopes on a B cell can be used in combination with a ligand conjugate such as those mentioned above, which are either cytotoxic to B cells by virtue of their biological effect, or by virtue of the cytotoxic agent fused or bound thereon.

Thus in additional embodiments, the antibody having specific binding for CDIM epitopes on a B cell can be used in a combined regimen with a ligand conjugate such as diphtheria toxin-conjugated IL-13, for example. The ligand conjugate can also comprise a radioactive isotope or other toxin, for example, to render it cytotoxic.

In additional embodiments, the antibody having specific binding for CDIM epitopes on a B cell is used to treat autoimmune disease in combination with a cytotoxic agent. The cytotoxic agent can be an immunosuppressant, such as a glucocorticoid, a calcineurin inhibitor, an antiproliferative/antimetabolic agent, or biologic agent such as an antibody that provides an immunosuppressant effect, or mixtures thereof. The combination with an immunosuppressant is useful in the treatment of autoimmune diseases mediated by B cells, or in some instances, for treating cancer. In particular embodiments, the calcineurin inhibitor is cyclosporine or tacrolimus. In other embodiments, the antiproliferative/antimetabolic agent is azathioprine, chlorambucol, cyclophosphamide, leflunomide, mycophenolate mofetil, methotrexate, rapamycin, thalidomide, or mixtures thereof. Glucocorticoids include, for example, prednisolone, prednisone, or dexamethasone.

In certain embodiments, the immunosuppressant is a cell growth regulator and/or inhibitor, which can include a small molecule therapeutic agent, gene therapy agent or gene expression modifier. Small molecule therapeutic agents include, for example, kinase inhibitors, and proteasome inhibitors. In a preferred embodiment, the kinase inhibitor is a bcr/abl tyrosine kinase inhibitor, such as GLEEVEC. In another preferred embodiment, the proteasome inhibitor is a boronic ester such as VELCADE.

In particular embodiments, the cytotoxic agent is a toxin, including without limitation Pseudomonas exotoxin A, ricin, diphtheria toxin, momordin, pokeweed antiviral protein, Staphylococcal enterotoxin A, gelonin, maytansinoids, daunarubicin, or the like. Preferably the toxin is conjugated to an antibody or ligand for cell specific targeting.

In a preferred embodiment, the condition characterized by a hyperproliferation of B cells is a lymphoid cancer, particularly any acute leukemia of B cell origin. Lymphoid cancers include acute leukemias, such as acute lymphocytic leukemia (ALL), B progenitor ALL, adult ALL, as well as chronic leukemias, and lymphomas. Lymphomas include aggressive, indolent and mantel cell types. Particular examples of lymphoid cancer include without limitation acute lymphocytic leukemia (ALL), non-Hodgkins lymphoma (NHL), Burkitt's lymphoma, B progenitor ALL, adult ALL, or chronic lymphocytic leukemia (CLL), and the like.

In particular embodiments, contacting hyperproliferating B cells can be performed in vivo, in vitro or ex vivo. Preferably, the B cells are contacted in vivo by administering said antibody having specific binding for CDIM epitopes on a B cell by parenteral injection. The in vivo contacting of B cells by the cytotoxic agent can be by any suitable means, as appropriate to the cytotoxic agent and its formulation, as known in the art.

In an additional aspect of the invention, there is provided a method of treating a human patient suffering from lymphoid cancer, comprising administering (1) a cytotoxic amount of an antibody having specific binding for CDIM epitopes on a B cell, and (2) a chemotherapeutic agent. In a preferred embodiment, the chemotherapeutic agent is a taxane, colchicine, vinca alkaloid, asparaginase, anti-actin agent, epipodophyllotoxin, camptothecin, antibiotic, platinum coordination complex, alkylating agent, folic acid analog, pyrimidine analog, purine analog or topoisomerase inhibitor, or mixtures thereof. In particular embodiments, the vinca alkaloid is vinblastine, vincristine, vindesine, or vinorelbine. Pyrimidine analogs include capecitabine, 5-fluoruracil, 5-fluorodeoxyuridine, 5-fluorodeoxyuridine monophosphate, cytosine arabinoside, 5-azacytidine, or 2′, 2′-difluorodeoxycytidine. The purine analog can be mercaptopurine, azathioprene, thioguanine, pentostatin, erythrohydroxynonyladenine, cladribine, vidarabine, or fludarabine phosphate. The folic acid analog can be methotrexate, raltitrexed, lometrexol, permefrexed, or edatrexate, pemetrexed. The epipodophyllotoxin can be etoposide or teniposide. Camptothecins include irinotocan, topotecan, camptothecan. Chemotherapeutic antibiotics include dactinomycin, daunorubicin, doxorubicin, idarubicin, epirubicin, valrubucin, mitoxanthrone, bleomycin, or mitomycin. Platinum coordination complexes include cisplatin, carboplatin, or oxaliplatin. Alkylating agents include mechlorethamine, cyclophosphamide, ifosfamide, melphalan, dacarbazine, temozolomide, thiotepa, hexamethylmelamine, streptozocin, carmustine, busulfan, altretamine or chlorambucil. Equivalents, modifications, and derivatives and the like are included within the scope of the chemotherapeutic agents that can be used in the methods and compositions of the invention. The chemotherapeutic agent can be administered before, after or simultaneously with the antibody having specific binding for CDIM epitopes.

In preferred embodiments, the antibody having specific binding for CDIM epitopes on a B cell comprises a CDR sequence having a net positive charge. In particular embodiments, the antibody having specific binding for CDIM epitopes on a B cell is a VH4-34 encoded antibody, including, without limitation, mAb 216, RT-2B, FS 12, A6(H4C5), Cal4G, S20A2, FS 3, Gee, HT, Z2D2, Y2K. A particularly preferred antibody is mAb 216.

In yet another aspect of the invention, there is provided a method of treating a human patient suffering from lymphoid cancer, comprising administering (1) a cytotoxic amount of an antibody having specific binding for CDIM epitopes on a B cell, and (2) a cytotoxic antibody having specific binding for a cell surface receptor on a B cell. In particular embodiments, the cytotoxic antibody can have specific binding for any cell surface molecule on a B cell (other than the CDIM epitope). For example, the cytotoxic antibody can exhibit specific binding for CD11 a, CD19, CD20, CD21, CD22, CD25, CD34, CD37, CD38, CD40, CD45, CD52, CD80, CD 86, IL-4R, IL-6R, IL-8R, IL-13, IL-13R, α-4 /β-1 integrin (VLA4), BLYS receptor, cell surface idiotypic Ig, tumor necrosis factor (TNF), or mixtures thereof, without limitation. For example, the cytotoxic antibody having specific binding for CD11a can be, for example, efalizumab (RAPTIVA). The cytotoxic antibody having specific binding for CD20 can be rituximab (RITUXAN). The cytotoxic antibody having specific binding for CD22 can be, for example, epratuzumab. The cytotoxic antibody having specific binding for CD25 can be, for example, daclizumab (ZENAPAX) or basiliximab (SIMULECT). Antibodies to CD52 include, e.g., CAMPATH. Antibodies to α-4/β-1 integrin (VLA4) include, e.g., natalizumab. Antibodies to TNF include, for example, infliximab (REMICADE). Thus in preferred embodiments, the antibody having specific binding for CDIM epitopes on a B cell can be used in a combined immunotherapy regimen with RITUXAN, ZENAPAX, REMICADE or RAPTIVA, for example, or in combinations thereof. The cytotoxic antibody can also be used as an immunoconjugate comprising a radioactive isotope or toxin, for example.

The antibody having specific binding for CDIM epitopes on a B cell comprises a CDR sequence having a net positive charge. In particular embodiments, the antibody having specific binding for CDIM epitopes on a B cell is a VH4-34 encoded antibody. Preferred VH4-34 antibodies include mAb 216, RT-2B, FS 12, A6(H4C5), Cal-4G, S20A2, FS 3, Gee. HT, Z2D2, Y2K.

In an additional embodiment, the method of treating a human patient suffering from lymphoid cancer comprising administering a cytotoxic amount of an antibody having specific binding for CDIM epitopes on a B cell, and a cytotoxic antibody having specific binding for a cell surface receptor on a B cell further comprises administering a chemotherapeutic agent, a radioactive isotope, an immunoconjugate, a ligand conjugate, an immunosuppressant, a cell growth regulator and/or inhibitor, or mixtures thereof. The antibody having specific binding for CDIM epitopes on a B cell can be labeled with a radioactive isotope. In addition, the cytotoxic antibody having specific binding for a cell surface receptor on a B cell can be labeled with a radioactive isotope. Preferred radioactive isotopes include ¹³¹I, ¹²⁵I, ⁹⁰Y, ¹¹¹In, and ¹⁸⁶Re. Either antibody can be used as an immunoconjugate. In preferred embodiments, the immunoconjugate comprises Pseudomonas exotoxin A, ricin, diphtheria toxin, momordin, pokeweed antiviral protein, Staphylococcal enterotoxin A, gelonin, maytansinoids, daunarubicin, or the like.

Ligand conjugates can comprise IL-2, IL-4, IL-6, IL-13, IL-15, BLYS, or TNF, and the like, and can further comprise a radioactive isotope, or a toxin. Immunosuppressants include glucocorticoids, calcineurin inhibitors, antiproliferative/antimetabolic agents or an antibodies, without limitation. Particular calcineurin inhibitors include cyclosporine, or tacrolimus, or the like. Particular antiproliferative/antimetabolic agents include azathioprine, chlorambucol, cyclophosphamide, leflunomide, mycophenolate mofetil, methotrexate, rapamycin, thalidomide, or mixtures thereof. Glucocorticoids can also be utilized, such as prednisolone, prednisone, or dexamethasone. Cell growth regulators and/or inhibitors include a small molecule therapeutic agent (e.g., a kinase inhibitor, or a proteasome inhibitor), gene therapy agent or gene expression modifier.

In another aspect of the invention, a method is provided of augmenting the B cell cytotoxicity of an antibody that binds a CDIM epitope, comprising contacting B cells with the antibody that binds a CDIM epitope and an agent that disrupts the cytoskeleton of B cells. Preferably, the agent that disrupts the cytoskeleton of B cells is an agent that interferes with the polymerization or depolymerization of microtubules, such as a taxane, vinca alkaloid or colchicine. Vinca alkaloids include vinblastine, vincristine, vindesine, or vinorelbine. Taxanes include without limitation paclitaxel, or docetaxel. The agent that disrupts the cytoskeleton of B cells can also be an anti-actin agent, i.e., an agent that affects actin filaments, either to polymerize actin or to depolymerize actin. In a preferred embodiment, the method of augmenting B cell cytotoxicity is used in the therapy of lymphoid cancer, B cell hyperproliferative diseases, or autoimmune diseases. Lymphoid cancer includes any acute leukemia of B cell origin, such as acute lymphocytic leukemia (ALL), non-Hodgkins lymphoma (NHL), Burkitt's lymphoma, B progenitor ALL, adult ALL, or chronic lymphocytic leukemia (CLL). In a preferred embodiment, the B cells are contacted by parenteral injection of a pharmaceutical formulation comprising a cytotoxic amount of the antibody that binds a CDIM epitope.

In yet another aspect, a method of treating an autoimmune disease in a mammal is provided, comprising administering (1) a cytotoxic amount of an antibody having specific binding for CDIM epitopes on a B cell, and (2) a chemotherapeutic agent, an antibody having specific binding for cell surface receptors on a B cell, an immunosuppressant, a cell growth regulator and/or inhibitor, or mixtures thereof. Preferably, the immunosuppressant is a glucocorticoid, a calcineurin inhibitor, or an antiproliferative/antimetabolic agent. Preferably, the calcineurin inhibitor is cyclosporine, or tacrolimus. The antiproliferative/antimetabolic agent can be azathioprine, chlorambucol, cyclophosphamide, leflunomide, mycophenolate mofetil, methotrexate, rapamycin, thalidomide, or mixtures thereof. The glucocorticoid can be selected from prednisolone, prednisone, or dexamethasone. The cell growth regulator and/or inhibitor can be a small molecule therapeutic agent, or a gene therapy agent or gene expression modifier.

Preferably, the antibody having specific binding for CDIM epitopes on a B cell comprises a CDR sequence having a net positive charge. In particular embodiments, the antibody having specific binding for CDIM epitopes on a B cell is a VH4-34 encoded antibody, such as mAb 216, RT-2B, FS 12, A6(H4C5), Cal4G, S20A2, FS 3, Gee, HT, Z2D2, Y2K. The method is useful for treating autoimmune diseases such as systemic lupus erythematosis, rheumatoid arthritis, autoimmune lymphoproliferative disease, multiple sclerosis, psoriasis, myasthenia gravis, Hashimoto's thyroiditis, lupus nephritis, dermatomyositis, Sjogren's syndrome, Sydenham's chorea, Alzheimer's disease, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, Crohn's disease, sarcoidosis, ulcerative colitis, erythema multiforme, IgA nephropathy, polyarteritis nodosa, ankylosing spondylitis, Goodpasture's syndrome, thromboangitis ubiterans, primary biliary cirrhosis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, fibrosing alveolitis, Class III autoimmune diseases such as immune-mediated thrombocytopenias, such as acute idiopathic thrombocytopenic purpura and chronic idiopathic thrombocytopenic purpura, and the like.

In another aspect of the invention, a method is provided for killing malignant B cells that are resistant to chemotherapeutic agents, cell growth regulators and/or inhibitors, or cytotoxic antibodies, comprising contacting said malignant B cells with an antibody having specific binding for CDIM epitopes on a B cell. In a particular embodiment, the method further comprises contacting the malignant B cells with a chemotherapeutic agent. In certain embodiments, the antibody is effective at a lower concentration than in the absence of the chemotherapeutic agent, and/or the chemotherapeutic agent is effective at a lower concentration than in the absence of the antibody.

In an additional aspect of the invention, a method is provided for killing malignant B cells that are resistant to an antibody having specific binding for CDIM epitopes on a B cell, comprising treating said B cells with a chemotherapeutic agent and/or an antibody having specific binding for CDIM epitopes on the B cells. In certain embodiments, the chemotherapeutic agent is effective at a lower concentration than in the absence of the antibody.

In additional aspects of the invention, a method of permeabilizing B cells is provided, comprising contacting B cells with an antibody having specific binding for CDIM epitopes on a B cell. The antibody having specific binding for CDIM epitopes on a B cell comprises a CDR sequence having a net positive charge. In preferred embodiments, the antibody having specific binding for CDIM epitopes on a B cell is a VH4-34 encoded antibody, such as mAb 216, RT-2B, FS 12, A6(H4C5), Cal-4G, S20A2, FS 3, Gee, HT, Z2D2, Y2K.

In yet another aspect of the invention, a method is provided for treating a disease or disorder characterized by a hyperproliferation of B cells, comprising contacting the B cells with an amount of an antibody having specific binding for CDIM epitopes on a B cell sufficient to permeabilize the B cells. The method can further comprise contacting said B cells with a cytotoxic agent. In particular embodiments, the step of contacting the B cells with the cytotoxic agent is performed before, during or after the step of contacting the B cells with antibody having specific binding for CDIM epitopes. The permeabilization of the B cells enhances efficacy of the cytotoxic agents by various means, and in certain embodiments, the efficacy of the cytotoxic agents is enhanced by increasing access of the cytotoxic agents to the cytosol of the B cell. In preferred embodiments, the cytotoxic agent is a chemotherapeutic agent, an immunosuppressant, a cell growth regulator and/or inhibitor, a toxin, or mixtures thereof. In additional preferred embodiments, the step of contacting the B cells is performed by parenterally injecting the antibody having specific binding for CDIM epitopes on a B cell into a human patient.

In particular aspects, the antibody having specific binding for CDIM epitopes on a B cell is administered at a dose of from about 2.5 to about 3000 mg/m², or more preferably, the dose of antibody administered is from about 25 to 1000 mg/m², or in particular, about 75, 150, 300 or 600 mg/m². In additional aspects, the antibody is administered at a dose of from about 0.25 mg/kg to about 100 mg/kg, and more preferably the dose of antibody administered is about 1.25, 2.5, 5, 10, or 20 mg/kg. The anti-CDIM antibody is typically administered on a weekly basis, and in some embodiments, more frequently than once per week, as often as once per day. Additional cytotoxic antibodies can be administered in an amount of 10-375 mg/m² per week for four weeks, or 0.4-20 mg/kg per week for 2 to 10 weeks.

In an additional aspect of the invention, a pharmaceutical formulation for parenteral injection is provided comprising a cytotoxic amount of an antibody having specific binding for CDIM epitopes on a B cell. In particular embodiments, the pharmaceutical formulation further comprises a chemotherapeutic agent.

In another aspect of the invention, a kit is provided for treating a patient suffering from a condition characterized by a hyperproliferation of B cells comprising: (a) pharmaceutical composition comprising an amount of an antibody having specific binding for CDIM epitopes on a B cell sufficient to permeabilize the B cells in the patient, and (b) a pharmaceutical composition comprising a therapeutically effective amount of a cytotoxic agent effective to treat the condition characterized by the hyperproliferation of B cells. Optional pharmaceutically acceptable solutions for injection for formulating the compositions can be provided. The antibody composition is preferably administered parenterally, and the cytotoxic agent can be administered by any means suitable. Instructions for administering the antibody composition and the cytotoxic agent composition can also be provided with the kit.

In an additional aspect, the invention includes the use of an antibody having specific binding for CDIM epitopes on a B cell in the manufacture of a medicament for treatment of B cell lymphoid cancers, autoimmune diseases and B cell hyperproliferative disorders.

Additional objects, advantages and novel features of the invention will be set forth in part in the description which follows, and in part will become apparent to those skilled in the art upon examination of the following, or may be learned by practice of the invention.

### BRIEF DESCRIPTION OF THE DRAWINGS

FIG. 1 illustrates that VH 4-34 encoded antibodies bind primary B cell lymphomas and leukemias.

FIG. 2 illustrates that VH4-34 encoded monoclonal antibodies bind and kill human B cell lines.

FIG. 3 illustrates the variability of the cytotoxicity of mAb 216 to follicular lymphoma cells.

FIG. 4 illustrates that the killing of B cells by mAb 216 and vincristine is synergistic.

FIG. 5A illustrates the time course of the appearance of Lamp-1 on the surface of B cells treated with mAb 216 compared with the time course of the loss of cell viability.

FIG. 5B illustrates the time course of release of ATP from damaged cells compared with the number of viable cells.

FIG. 6A illustrates the viability of cells treated with two VH4-34 antibodies in medium with and without calcium.

FIG. 6B illustrates the viability of cells treated with cytotoxic agents.

FIG. 7 illustrates the efficacy for killing cells by C2B8, mAb 216 and the combination of the two antibodies, at two different cell concentrations.

### DETAILED DESCRIPTION OF THE INVENTION

I. Definitions and Overview

Before the present invention is described in detail, it is to be understood that unless otherwise indicated this invention is not limited to particular buffers, excipients, chemotherapeutic agents, or the like, as such may vary. It is also to be understood that the terminology used herein is for the purpose of describing particular embodiments only and is not intended to limit the scope of the present invention.

It must be noted that as used herein and in the claims, the singular forms “a,” “and” and “the” include plural referents unless the context clearly dictates otherwise. Thus, for example, reference to “a chemotherapeutic agent” includes two or more chemotherapeutic agents; reference to “a pharmaceutical excipient” includes two or more pharmaceutical excipients, and so forth.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The terms “anti-CDIM antibody” and “CDIM binding antibody” as used herein refers to an antibody having specific binding for CDIM epitopes on a B cell. These terms will be used interchangeably herein.

An agent which “arrests the growth of” or a “growth inhibitory agent” as used herein refers to a compound or composition which inhibits growth or proliferation of a cell, especially a neoplastic cell type expressing a B cell antigen such as the CD20 antigen as required. Thus, the growth inhibitory agent is one which for example, significantly reduces the percentage of neoplastic cells in S phase.

The terms “cancer” and “cancerous” refer to or describe the physiological condition in mammals that is typically characterized by unregulated cell growth.

The “CD20” antigen is a 35 kDa, non-glycosylated phosphoprotein found on the surface of greater than 90% of B cells from peripheral blood or lymphoid organs. CD20 is expressed during early pre-B cell development and remains until plasma cell differentiation. CD20 is present on both normal B cells as well as malignant B cells. Other names for CD20 in the literature include “B-lymphocyte-restricted antigen” and “Bp35.” The CD20 antigen is described in Clark et al. PNAS (USA) 82:1766 (1985), for example.

The term “cell wounding” refers to a survivable plasma membrane disruption event marked by the uptake into the cytosol of a normally membrane impermeant tracer. Cell wounding disruptions typically are in the range of between about 1 and 1000 μm², and thus are far larger than the membrane disruptions accompanying complement mediated cytotoxicity or perforin or even large pores formed by toxins or pore forming agents such as gramicidin or Staphylococcus aureus alpha toxin. Cell wounding is detected by the cellular repair mechanism manifested as a result of the wound, namely the expression of Lamp-1 on the cellular surface as a result of lysosomal fusion to repair the wound.

The term “chemotherapeutic agent” refers to a chemical compound useful in the treatment of cancer or other condition characterized by a hyperproliferation of cells.

The terms “cytotoxic agent” and “cytotoxin” as used herein refer to a substance that inhibits or arrests the growth of, inhibits or prevents the function of cells, and/or causes death of cells. The term is intended to include one or more radioactive isotopes, chemotherapeutic agents, immunosuppressants, cell growth regulators and/or inhibitors, which can be small molecule therapeutics, cytotoxic antibodies, and toxins such as enzymatically active toxins of bacterial, fungal, plant or animal origin, or fragments thereof. The term also includes immunoconjugates comprising antibodies labeled with toxins or radioactive isotopes for specific binding to a target cell, as well as other ligand conjugates, such as radiolabeled ligands, and toxin-labeled ligands. In addition, one or more cytotoxic agents can be used in combination.

A “disorder” is any condition that would benefit from treatment with the combination therapy described herein. This includes chronic and acute disorders or diseases including those pathological conditions which predispose the mammal to the disorder in question. Non-limiting examples of disorders to be treated herein include cancer, hematological malignancies, leukemias and lymphoid malignancies and autoimmune diseases such as inflammatory and immunologic disorders.

The terms “hyperproliferation” and “hyperproliferating” refer to the abnormal growth of a cell type, which can be cancerous or benign. Hyperproliferation includes the polyclonal expansion of B cells secreting autoantibodies that mediate autoimmune diseases.

The term “immunoconjugates” refers to antibodies conjugated to cytotoxic agents, which can be covalent or noncovalently associated.

The term “intravenous infusion” refers to introduction of an agent into the vein of an animal or human patient over a period of time, generally greater than approximately 15 minutes, and more generally between approximately 30 to 90 minutes.

The term “intravenous bolus” or “intravenous push” refers to drug administration into a vein of an animal or human such that the body receives the drug in approximately 15 minutes or less, generally 5 minutes or less.

The term “mammal” for purposes of treatment refers to any mammalian species, including humans, domestic and farm animals, and zoo, sports, or pet animals, so long as the CDIM antigen expression is predominantly restricted to cells of B cell lineage, after birth.

The humanized anti-CD20 antibody referred to as the “RITUXAN® brand” anti-CD20 antibody is a genetically engineered chimeric murine/human monoclonal antibody directed against the CD20 antigen. Rituximab is the antibody called “C2B8” in U.S. Pat. No. 5,736,137 issued Apr. 7, 1998. The RITUXAN® brand of C2B8 antibody is indicated for the treatment of patients with relapsed or refractory low-grade or follicular, CD20 positive, B cell non-Hodgkin's lymphoma.

The term specific binding refers the property of having a high binding affinity of at least 10⁶ M⁻¹, and usually between about 10⁶ M⁻¹ and about 10⁸ M⁻¹.

The term “subcutaneous administration” refers to introduction of an agent under the skin of an animal or human patient, preferable within a pocket between the skin and underlying tissue, by relatively slow, sustained delivery from a drug receptacle. The pocket may be created by pinching or drawing the skin up and away from underlying tissue.

The term “subcutaneous bolus” refers to drug administration beneath the skin of an animal or human patient, where bolus drug delivery is preferably less than approximately 15 minutes, more preferably less than 5 minutes, and most preferably less than 60 seconds. Administration is preferably within a pocket between the skin and underlying tissue, where the pocket

The term “subcutaneous infusion” refers to introduction of a drug under the skin of an animal or human patient, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the animal or human patient, wherein the pump delivers a predetermined amount of drug for a predetermined period of time, such as 30 minutes, 90 minutes, or a time period spanning the length of the treatment regimen.

The term “therapeutically effective amount” is used to refer to an amount of an active agent having a growth arrest effect or causes the death of the cell. In certain embodiments, the therapeutically effective amount has the property of permeabilizing cells, inhibiting proliferative signaling, inhibiting cellular metabolism, promoting apoptotic activity, or inducing cell death. In particular aspects, the therapeutically effective amount refers to a target serum concentration that has been shown to be effective in, for example, slowing disease progression. Efficacy can be measured in conventional ways, depending on the condition to be treated. For example, in lymphoid cancers, efficacy can be measured by assessing the time to disease progression (TTP), or determining the response rates (RR).

The terms “treat,” “treatment” and “therapy” and the like as used within the context of the present invention, are meant to include therapeutic as well as prophylactic, or suppressive measures for a disease or disorder leading to any clinically desirable or beneficial effect, including but not limited to alleviation of one or more symptoms, regression, slowing or cessation of progression of the disease or disorder. Thus, for example, the term treatment includes the administration of an agent prior to or following the onset of a symptom of a disease or disorder thereby preventing or removing all signs of the disease or disorder. As another example, the term includes the administration of an agent after clinical manifestation of the disease to combat the symptoms of the disease. Further, administration of an agent after onset and after clinical symptoms have developed where administration affects clinical parameters of the disease or disorder, such as the degree of tissue injury or the amount or extent of metastasis, whether or not the treatment leads to amelioration of the disease, comprises “treatment” or “therapy' within the context of the invention.

The VH4-34 gene (variable heavy region) is one of the 53 identified human functional antibody germline genes ¹. The VH4-34 gene is present in all haplotypes and no sequence variation has been reported in germline DNA isolated from unrelated individuals ^{2 3}. Antibodies encoded by the VH4-34 gene have been shown to possess unique properties. All mAbs directed against the “I” or “i” antigens of red blood cells (RBCs) are encoded by the VH4-34 gene ^{4 5 6}, are generally of the IgM class, and are classically described as cold agglutinins (CAs) because they agglutinate RBCs at 4° C. The ligands recognized by CAs are linear or branched glycoconjugates present on proteins and/or lipids of the RBCs. Newborn and cord blood RBC possess the linear i antigen. The branched I chain is generated after birth ^{7 8}. The “i” antigen recognized on human B cells is a linear lactosamine determinant that is sensitive to the enzyme endo-beta-galactosidase. Sequence analysis of independently derived VH4-34 anti-B cell/anti-i mAbs has shown that they are in germline configuration but express independent D, J, H, and light chains ²⁰.

In vivo, the expression of VH4-34 gene derived antibodies is strictly regulated. Although 4-8% of human B cells express VH4-34 encoded antibody, serum levels of VH4-34 derived antibodies are negligible in normal adults ^{9 10}. Increase in circulating VH4-34 derived antibodies is seen only in selective pathological conditions including EBV (mononucleosis) and HIV infection and certain autoimmune diseases ^{11 12 13 14 15 16}.

The present inventors have extensively studied VH4-34 encoded antibodies and their role in autoimmune disorders. Previous studies demonstrated that certain anti-B cell VH4-34 antibodies are cytotoxic to B cells and lead to decreased B cell proliferation Bhat, N. et al. (1997) Clin. Exp. Immunol. 108:151; Bhat, N., et al., (2001) Crit. Rev. Oncol. Hematol. 39:59. Cytotoxicity was shown to be independent of complement, and to be highly temperature dependent, resulting in greater cell death and the formation of plasma membrane defects such as blebs and pores on the cell surface when treated at 4° C. The plasma membrane defects were shown to be significantly larger than the pores formed by other well known pore-forming proteins, such as C9 complement component (˜100 Å) and perforin (˜160 Å). It was suggested that the cytotoxicity may be mediated by a novel mechanism.

The present inventors have made the surprising and unexpected discovery that these VH4-34 gene derived antibodies can induce cell membrane wounding in B cells. Although membrane injury is a common threat faced by nucleated mammalian cells, the fact that an antibody could be the direct cause of membrane injury is novel. In addition, the present inventors have discovered that although the antibody causes pores and membrane defects in cells under certain conditions, when treated at sublethal concentrations, some of the B cells are merely wounded, and are capable of repairing the wound in some cases.

Further, the present inventors have demonstrated that antibody induced cell membrane wounding is repaired in a manner similar to any other membrane wound. Cells treated with these complement independent cytotoxic antibodies attempt to repair the antibody induced cell membrane wound utilizing lysosomal fusion with the plasma membrane to patch the membrane wound, resulting in the appearance of lysosomal membrane proteins on the cell surface. It is also demonstrated that when the cells are unable to repair the damage, death ultimately results.

In addition, the present inventors have discovered that the wounded cells are permeabilized, at least transiently, and become more susceptible to the action of additional cytotoxic agents, providing novel treatment options having enhanced efficacy for treatment of human and animal diseases and disorders. The cell membrane wound results in permeabilization of the B cells and allows entry of cytotoxic agents such as chemotherapeutic agents, thus increasing the efficacy of the chemotherapeutic agents, even in cells that are resistant or impermeable to such agents, or in cells that actively transport them out of the cell.

Because the mechanism of cell death and wounding provided by the CDIM binding antibodies is different from the cytotoxic mechanism utilized by conventional cytotoxic antibodies (complement or cell mediated killing), the combination of the CDIM binding antibodies with conventional immunotherapies can provide an enhanced efficiency of killing by cytotoxic antibodies binding additional B cell antigens, especially under conditions of immunodeficiencies such as complement depletion or deficiency.

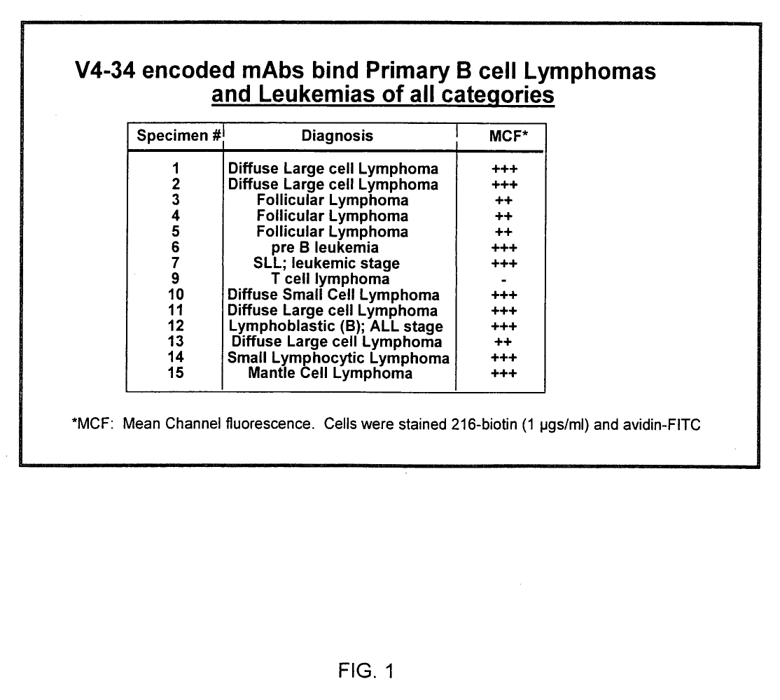

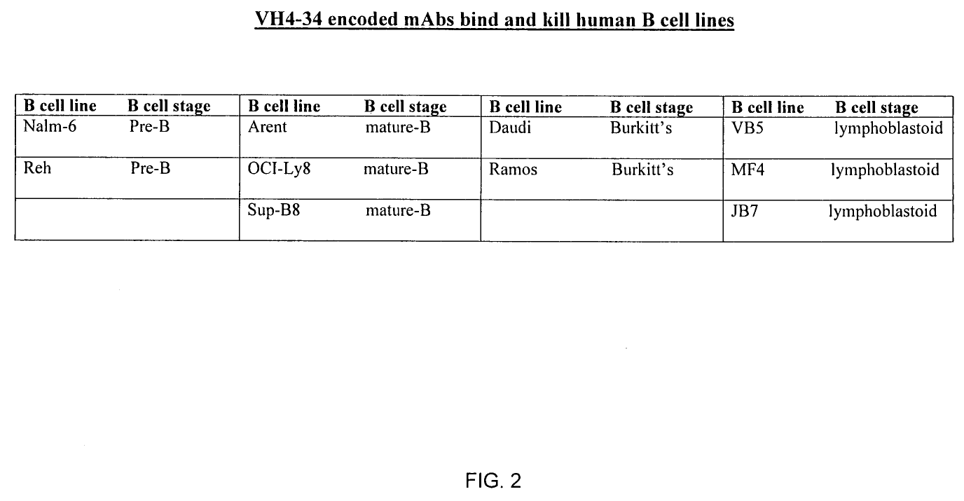

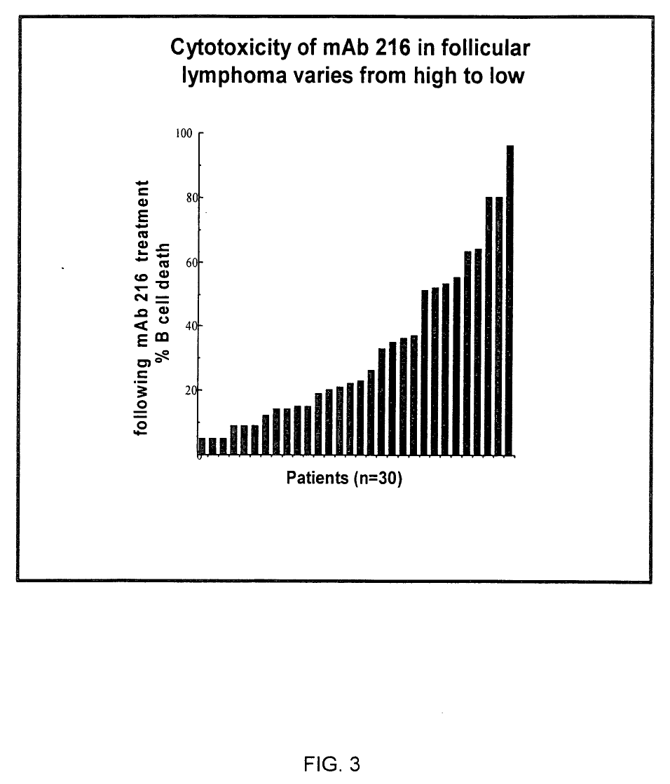

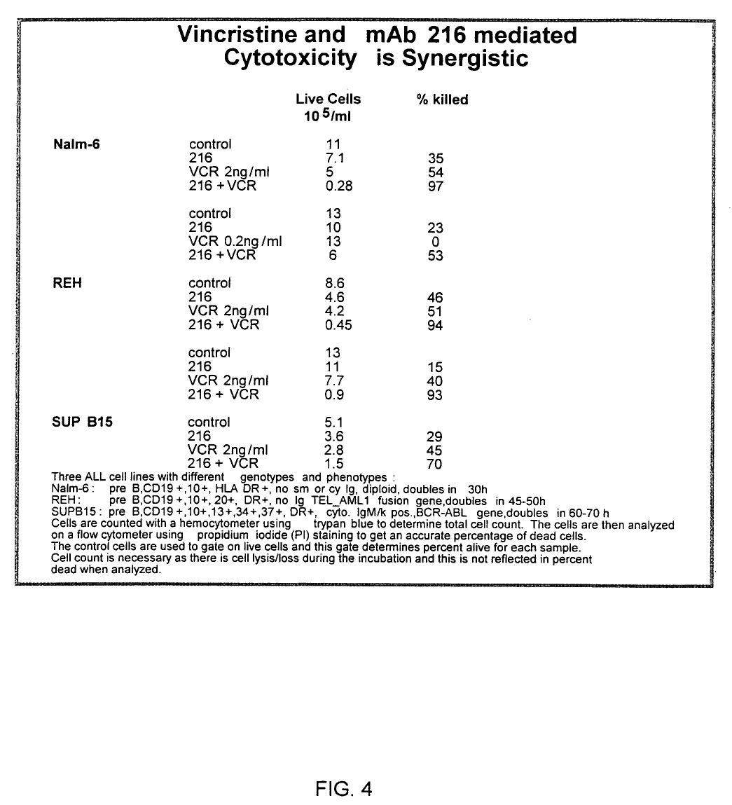

In a preferred embodiment, the antibodies according to one aspect of the invention are VH4-34 encoded monoclonal antibodies that bind the CDIM epitope on human B cells ^{17 18 19}, as illustrated in FIGS. 1 and 2. These antibodies are cytotoxic to B cells obtained from relapsed follicular lymphoma patients, as illustrated in FIG. 3. In addition, the antibodies are cytotoxic to B cell lines, as shown in FIG. 4. In a preferred embodiment, these mAbs are produced by fusion of human lymphocytes and a heteromyeloma cell line, which produces a hybridoma secreting human antibody. For example, mAb 216 is a human IgM encoded by the VH4-34 gene, and is a preferred embodiment of the CDIM binding VH4-34 antibodies described herein. MAb 216 is further described in U.S. Pat. Nos. 5,593,676 and 5,417,972 and EP 712 307 B1 to Bhat, et al.

Additional VH4-34 derived antibodies that bind the CDIM epitope include RT-2B, FS 12, A6(H4C5), Cal-4G, S20A2, FS 3, Gee, HT, Z2D2, Y2K. Certain of these antibodies are characterized by a CDR3 sequence rich in basic amino acid residues, and by particularly strong binding when the net charge of the CDR3 is +2. Accordingly, any antibody possessing a net positive CDR, particularly CDR3, and exhibiting binding to the CDIM epitope, is encompassed within the scope of the invention and as claimed in the appended claims.

The present inventors have made the surprising discovery that the B cell toxicity of these anti-CDIM antibodies can be markedly and even synergistically enhanced by the addition of a cytotoxic agent, including chemotherapeutic agents, radioactive isotopes, cytotoxic antibodies, immunoconjugates, ligand conjugates, immunosuppressants, cell growth regulators and/or inhibitors, toxins, or mixtures thereof.

Accordingly, in one embodiment, a method of treating a mammal suffering from a condition characterized by hyperproliferation of B cells is provided, comprising contacting said B cells with (1) a cytotoxic amount of an antibody having specific binding for CDIM epitopes on a B cell, and (2) a cytotoxic agent. Hyperproliferation of B cells occurs in patients suffering from cancer, viral diseases, immunodeficiencies or autoimmune diseases.

In yet another aspect of the invention, a method is provided for treating a disease or disorder characterized by a hyperproliferation of B cells, comprising contacting the B cells with an amount of an antibody having specific binding for CDIM epitopes on a B cell sufficient to permeabilize the B cells. The method can further comprise contacting said B cells with a cytotoxic agent.

Treatment of Lymphoid Cancers

The antibody having specific binding for CDIM epitopes on a B cell can be used to treat the hyperproliferation of B cells that occurs in any lymphoid cancer, particularly any acute leukemia of B cell origin. Lymphoid cancers include acute leukemias, such as acute lymphocytic leukemia (ALL), B progenitor ALL, adult ALL, as well as chronic leukemias, and lymphomas. Lymphomas include non-Hodgkins lymphoma (NHL), and aggressive, indolent and mantel cell types. The lymphoid cancers can include peripheral as well as central nervous system lymphomas, follicular lymphomas, mucosal lymphomas, without limitation. Particular examples of lymphoid cancer include, without limitation, acute lymphocytic leukemia (ALL), non-Hodgkins lymphoma (NHL), Burkitt's lymphoma, B progenitor ALL, adult ALL, or chronic lymphocytic leukemia (CLL), and the like.

A representative treatment protocol is set forth in Example 11 for treating ALL. Additional chemotherapeutic treatment regimens can be utilized in combination with anti-CDIM antibodies for the treatment of ALL or other lymphoid cancers of B cell origin, and these additional chemotherapeutic treatment regimens are included within the scope of the invention without limitation.

Treatment of B cell Hyperproliferation Due to Viral Diseases

The antibody having specific binding for CDIM epitopes on a B cell can be used to treat B Cell Hyperproliferation that occurs in certain viral infections such as human immunodeficiency virus or mononucleosis.

Treatment of B Cell Hyperproliferation Due to Immunodeficiencies

The antibody having specific binding for CDIM epitopes on a B cell can be used to treat B cell hyperproliferation occuring in certain immune deficiencies occurring as a result of cancer therapies or immunosuppressive therapies to treat autoimmune disorders. For example, B cell hyperproliferation occurs in post-transplant lymphoproliferative disease and immunodeficiency syndrome in patients receiving anticancer therapies or other immunosuppressive therapies.

Treatment of Autoimmune Disease Mediated by B Cells

The antibody having specific binding for CDIM epitopes on a B cell can be used to treat autoimmune disease, either alone or in combination with a cytotoxic agent. The cytotoxic agent can be an immunosuppressant, such as a glucocorticoid, a calcineurin inhibitor, an antiproliferative/antimetabolic agent, or biologic agent such as an antibody that provides an immunosuppressant effect, or mixtures thereof. The combination with an immunosuppressant is useful in the treatment of autoimmune diseases mediated by B cells, or in some instances, for treating cancer. In particular embodiments, the calcineurin inhibitor is cyclosporine or tacrolimus. In other embodiments, the antiproliferative/antimetabolic agent is azathioprine, chlorambucol, cyclophosphamide, leflunomide, mycophenolate mofetil, methotrexate, rapamycin, thalidomide, or mixtures thereof. Glucocorticoids include, for example, prednisolone, prednisone, or dexamethasone.

In certain embodiments, the immunosuppressant is a cell growth regulator and/or inhibitor, which can include a small molecule therapeutic agent, gene therapy agent or gene expression modifier. Small molecule therapeutic agents include, for example, kinase inhibitors, and proteasome inhibitors. In a preferred embodiment, the kinase inhibitor is a bcr/abl tyrosine kinase inhibitor, such as GLEEVEC. In another preferred embodiment, the proteasome inhibitor is a boronic ester such as VELCADE.

In particular embodiments, the cytotoxic agent is a toxin, including without limitation Pseudomonas exotoxin A, ricin, diphtheria toxin, momordin, pokeweed antiviral protein, Staphylococcal enterotoxin A, gelonin, maytansinoids, daunarubicin, or the like. Preferably the toxin is conjugated to an antibody or ligand for cell specific targeting.

Representative autoimmune diseases include systemic lupus erythematosis, rheumatoid arthritis, autoimmune lymphoproliferative disease, multiple sclerosis, psoriasis, and myasthenia gravis, but can also include Hashimoto's thyroiditis, lupus nephritis, dermatomyositis, Sjogren's syndrome, Alzheimer's Disease, Sydenham's chorea, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, Crohn's disease, sarcoidosis, ulcerative colitis, erythema multiforme, IgA nephropathy, polyarteritis nodosa, ankylosing spondylitis, Goodpasture's syndrome, thromboangitis ubiterans, primary biliary cirrhosis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis, fibrosing alveolitis, Class III autoimmune diseases such as immune-mediated thrombocytopenias, such as acute idiopathic thrombocytopenic purpura and chronic idiopathic thrombocytopenic purpura, and the like.

Methods for Reducing Tumor Load and Permitting Reinduction Therapy

By administering a cytotoxic amount of an antibody having specific binding for CDIM epitopes on a B cell to a patient, a method is provided for reducing tumor load in the patient suffering from lymphoid cancer prior to treatment with conventional chemo- or immunotherapy.

In addition, when the patient becomes refractory to conventional chemotherapeutic or immunotherapies and requires reinduction, the patient can be prepared for reinduction therapy by administering the antibody having specific binding for CDIM epitopes on a B cell, such as mAb 216. This treatment reduces the numbers of living tumor cells in the patient and allows the patient to undergo subsequent reinduction therapy.

In Vitro and Ex Vivo Uses

In another embodiment, the methods herein include a method for purging the bone marrow of a patient suffering from lymphoid cancer of malignant B cells prior to reimplantation of the bone marrow in the patient after myeloablative therapy. The method comprises treating the bone marrow of the patient ex vivo with a cytotoxic amount of an antibody having specific binding for CDIM epitopes on a B cell. The method can further comprise treating the bone marrow cells ex vivo with a cytotoxic agent such as a chemotherapeutic agent or cytotoxic antibody.

Prescreening Patient Cells In Vitro for Susceptibility to Anti-CDIM Antibodies

In one embodiment, a sample of a patient's blood can be tested for specific binding of antibodies to CDIM epitopes on B cells and antibody mediated cytotoxicity, preferably by a VH4-34 antibody such as mAb 216. For patients exhibiting less than 100% cell killing, combinations with additional cytotoxic agents can be tested for optimization of patient therapies.

Advantages of Treatment Methods

By administering a cytotoxic amount of an antibody having specific binding for CDIM epitopes on a B cell prior to, during or even after treatment with conventional immunotherapy, methods are provided for augmenting the cytotoxicity of cytotoxic agents utilized during chemotherapy and anti-B cell antibodies utilized during immunotherapy. Conventional anti-B cell immunotherapy may lack efficacy under conditions of high tumor load or immunodeficiency. For example, when complement stores are depleted, the anti-B cell immunotherapy can be rendered inefficacious. The combination with an antibody having specific binding for CDIM epitopes on a B cell can overcome this lack of efficacy of conventional anti-B cell immunotherapy, because the anti-CDIM antibody acts using a different mechanism of toxicity, inducing cell wounding. The cell wounding exacerbates any complement mediated membrane leakage due to the conventional immunotherapy. In combination with an additional cytotoxic agent, the anti-CDIM antibody can greatly augment the cytotoxicity of a therapeutic regimen, by increasing the cytosolic access of chemotherapeutic or other cytotoxic agents to the B cell cytosol.

In addition, many cancer or autoimmune disease patients are fragile and cannot tolerate aggressive therapies. The novel mechanism of action of the anti-CDIM antibody, especially when used in combination with chemotherapeutic agents, can allow for the treatment of fragile patients, for example, by increasing the efficacy of the treatment regimen, and by allowing the patient to be treated with lower doses of chemotherapeutic agents than would otherwise be efficacious.

Antibodies

Antibodies useful in the present invention include anti-CDIM antibodies and additional cytotoxic antibodies having specific binding for cell surface molecules on a B cell. The anti-CDIM antibodies and additional cytotoxic antibodies can be used in combination treatment regimen.

The cytotoxic antibody can have specific binding for any cell surface molecule on a B cell. Cell surface molecules include receptors, immunoglobulins, cytokines, glycoproteins, etc. For example, the cytotoxic antibody can exhibit specific binding for CD11a, CD19, CD20, CD21, CD22, CD25, CD34, CD37, CD38, CD40, CD45, CD52, CD80, CD 86, IL4R, IL-6R, IL-8R, IL-13, IL-13R, α-4/β-1 integrin (VLA4), BLYS receptor, cell surface idiotypic Ig, tumor necrosis factor (TNF),or mixtures thereof, without limitation. For example, the cytotoxic antibody having specific binding for CD1 la can be, for example, efalizumab (RAPTIVA). The cytotoxic antibody having specific binding for CD20 can be rituximab (RITUXAN). The cytotoxic antibody having specific binding for CD22 can be, for example, epratuzumab. The cytotoxic antibody having specific binding for CD25 can be, for example, daclizumab (ZENAPAX) or basiliximab (SIMULECT). Antibodies to CD52 include, e.g., CAMPATH. Antibodies to α-4/β1 integrin (VLA4) include, e.g., natalizumab. Antibodies to TNF include, for example, infliximab (REMICADE).

Thus in preferred embodiments, the antibody having specific binding for CDIM epitopes on a B cell can be used in a combined immunotherapy regimen with RITUXAN, ZENAPAX, REMICADE or RAPTIVA, for example, or in combinations thereof. The cytotoxic antibody can also be used as an immunoconjugate comprising a radioactive isotope or toxin, for example. Further, in additional embodiments, a combined therapy can be used comprising the antibody having specific binding for CDIM epitopes on a B cell, an additional cytotoxic antibody having specific binding for cell surface molecules on a B cell, and one or more chemotherapeutic agents. For example, mAb216 could be used in combination with an anti-CD20 antibody such as rituximab, tosutimab, or ibritumomab, with an anti-CD22 antibody, such as, epratuzumab, or in combination with an anti-CD52 antibody such as CAMPATH. The combination therapy can further include chemotherapy, such as an agent that disrupts the cytoskeleton of the cell, e.g., vincristine, in a combined chemotherapy and immunotherapy regimen.

The term “antibody” is used in the broadest sense and specifically covers intact natural antibodies, monoclonal antibodies, polyclonal antibodies, multispecific antibodies (e.g. bispecific antibodies) formed from at least two intact antibodies, synthetic antibodies such as tetravalent antibodies, and antibody fragments, so long as they exhibit the desired biological activity. Human antibodies include antibodies made in nonhuman species. The term antibody also encompasses fusion or chemical coupling of antibodies with cytotoxic or cell regulating agents.

“Antibody fragments” comprise a portion of an intact antibody, preferably the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062[1995]); single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855[1984]).

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat or rabbit having the desired specificity, affinity, and capacity. In some instances, framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDRs correspond to those of a non-human immunoglobulin and all or substantially all of the FRs are those of a human immunoglobulin sequence. The humanized antibody optimally also will comprise at least a portion of an immunoglobulin constant region (Fc), typically that of a human immunoglobulin. For further details, see Jones et al., (1986) Nature 321:522-525; Reichmann et al., (1988) Nature 332:323-329; and Presta, (1992) Curr. Op. Struct. Biol., 2:593-596. The humanized antibody includes a PRIMATIZED™ antibody wherein the antigen-binding region of the antibody is derived from an antibody produced by immunizing macaque monkeys with the antigen of interest.

“Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the sFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH—VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Immunoconjugates

Immunoconjugates can be prepared by numerous methods known in the art, such as chemical derivatization of the antibody to provide reactive crosslinking groups, which can be labile or non-labile. Labile reactive groups provide for the release of the cytotoxic agent or growth regulator from the antibody. Non-labile crosslinking is also useful. The linkage of the desired agent to the Ig molecule may be achieved by a variety of means known to the art including conventional coupling techniques (e.g., coupling with dehydrating agents such as dicyclohexylcarbodiimide (DCCI), ECDI and the like), the use of linkers capable of coupling through sulfhydryl groups, amino groups or carboxyl groups (available from Pierce Chemical Co., Rockford, Ill.), by reductive amination.

In one method, an antibody conjugate, or immunoconjugate, can be prepared by first modifying the antibody with a cross-linking reagent such as N-succinimidyl pyridyldithiopropionate (SPDP) to introduce dithiopyridyl groups into the antibody (Carlsson et al. (1978) Biochem. J. 173:723-737; U.S. Pat. No. 5,208,020). In a second step, a cytotoxin having a thiol group, is added to the modified antibody, resulting in the displacement of the thiopyridyl groups in the modified antibodies, and the production of disulfide-linked cytotoxin-antibody conjugate. A procedure to prepare maytansinoid-antibody conjugates is described in U.S. Pat. No. 5,208,020.

In some instances, fusion proteins of antibody and cytotoxic agents may be desired. Fusion proteins can be prepared by molecular biological means (e.g., the production of a fusion protein using an expression vector comprising a nucleotide sequence encoding the recombinant Ig operably linked to a nucleotide sequence encoding the desired cytotoxic agent).

Radioactive Isotopes

The isotopes used to produce therapeutically useful immuno- or ligand conjugates typically produce high energy α-, γ- or β-particles which have a therapeutically effective path length. Such radionuclides kill cells to which they are in close proximity, for example neoplastic cells to which the conjugate is bound. The advantage of targeted delivery is that the radioactively labeled antibody or ligand generally has little or no effect on cells not in the immediate proximity of the targeted cell.

With respect to the use of radioactive isotopes as cytotoxic agents, modified antibodies or ligands may be directly labeled (such as through iodination) or may be labeled using of a chelating agent. In either method, the antibody or ligand is labeled with at least one radionuclide. Particularly preferred chelating agents comprise l-isothiocyamatobenzyl-3-methyldiothelene triaminepentaacetic acid (“MX-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y.

The radioactive isotope can be attached to specific sites on the antibody or ligand, such as the N-linked sugar resides present only on the Fc portion of the antibody. Technetium-99m labeled antibodies or ligands may be prepared by ligand exchange processes or by batch labeling processes. For example, the antibody can be labeled by reducing pertechnate (TcO₄) with stannous ion solution, chelating the reduced technetium onto a Sephadex column and applying the antibody to this column. Batch labeling techniques include, for example, incubating pertechnate, a reducing agent such as SnCl₂, a buffer solution such as a sodium-potassium phthalate-solution, and the antibody. Preferred radionuclides for labeling are well known in the art. An exemplary radionuclide for labeling is ¹³¹I covalently attached via tyrosine residues. Radioactively labeled antibodies according to the invention can be prepared with radioactive sodium or potassium iodide and a chemical oxidizing agent, such as sodium hypochlorite, chloramine T or the like, or an enzymatic oxidizing agent, such as lactoperoxidase, glucose oxidase and glucose.

Patents relating to chelators and chelator conjugates are known in the art. For example, U.S. Pat. No. 4,831,175 to Gansow is directed to polysubstituted diethylenetriaminepentaacetic acid chelate and protein conjugates containing the same and methods for their preparation. U.S. Pat. Nos. 5,099,069, 5,246,692, 5,286,850, 5,434,287 and 5,124,471 all to Gansow also relate to polysubstituted DTPA chelates. These patents are incorporated herein by reference in their entireties. Other examples of compatible metal chelators are ethylenediaminetetraacetic acid (EDTA), diethylenetriaminepentaacetic acid (DPTA), 1,4,8,11 -tetraazatetradecane, 1,4,8,11 tetraazatetradecane- 1,4,8,11 -tetraacetic acid, 1-oxa-4,7,12,15-tetraazaheptadecane, 4,7, 12,15-tetraacetic acid, or the like. Cyclohexyl-DTPA or CHX-DTPA is particularly preferred. Still other compatible chelators, including those yet to be discovered, may easily be discerned by a skilled artisan and are clearly within the scope of the present invention. Additional chelators include the specific bifunctional chelators described in U.S. Pat. Nos. 6,682,734, 6,399,061 and 5,843,439, and are preferably selected to provide high affinity for trivalent metals, exhibit increased tumor-to-non-tumor ratios and decreased bone uptake as well as greater in vivo retention of radionuclide at target sites, i.e., B-cell lymphoma tumor sites. However, other bifunctional chelators that may or may not possess all of these characteristics are known in the art and may also be beneficial in tumor therapy.

Modified antibodies can also be conjugated to radioactive labels for diagnostic as well as therapeutic purposes. Radiolabeled therapeutic conjugates for diagnostic “imaging” of tumors can also be utilized before administration of antibody and cytotoxic agent to a patient. For example, the monoclonal antibody binding the human CD20 antigen known as C2B8 can be radiolabeled with ¹¹¹In using a bifunctional chelator, such as MX-DTPA (diethylenetriaminepentaacetic acid), which comprises a 1:1 mixture of 1-isothiocyanatobenzyl-3-methyl-DTPA and 1 -methyl-3-isothiocyanatobenzyl-DTPA. ¹¹¹In is a preferred diagnostic radioactive isotope since between about 1 and about 10 mCi can be safely administered without detectable toxicity, and the imaging data is an indicator of subsequent ⁹⁰Y-labeled antibody distribution. A typical dose of ¹¹¹In-labeled antibody of 5 mCi for imaging studies is used, and optimal imaging can be determined at various times after administration of the labeled antibody or ligand, typically three to six days after administration. See, for example, Murray, J. (1985) Nuc. Med. 26: 3328 and Carraguillo et al., (1985) J. Nuc. Med. 26: 67.

A variety of radioactive isotopes can be utilized and one skilled in the art can readily determine which radioactive isotope is most appropriate under various conditions. For example, ¹³¹I is frequently utilized for targeted immunotherapy. However, the clinical usefulness of ¹³¹I can be limited by its short half life (8 days), the potential for dehalogenation of iodinated antibody both in the blood and at tumor or sites, and its high energy γ emission which may not provide sufficiently localized dose deposition in tumor, depending on tumor size, as desired. With the advent of additional chelating agents, additional opportunities are provided for attaching metal chelating groups to proteins and utilizing other radionuclides such as ¹¹¹In and ⁹⁰Y. ⁹⁰Y provides several benefits for utilization in radioimmunotherapeutic applications. For example, the longer useful half life of 64 hours for ⁹⁰Y is sufficiently long to allow antibody accumulation by tumor cells and, unlike ¹³¹I, ⁹⁰Y is a pure beta emitter of high energy with no accompanying gamma radiation in its decay, having a range in tissue of 100 to 1,000 cell diameters. Furthermore, the minimal amount of penetrating radiation allows for outpatient administration of ⁹⁰Y-labeled antibodies. Additionally, internalization of labeled antibody is not required for cell killing, and the ionizing radiation should be lethal for adjacent tumor cells lacking the target antigen.

Effective single treatment dosages (i.e., therapeutically effective amounts) of ⁹⁰Y-labeled antibodies range from between about 5 and about 75 mCi, more preferably between about 10 and about 40 mCi. Effective single treatment non-marrow ablative dosages of ¹³¹I-labeled antibodies range from between about 5 and about 70 mCi, more preferably between about 5 and about 40 mCi. Effective single treatment ablative dosages (i.e., that may require autologous bone marrow transplantation) of ¹³¹I labeled antibodies range from between about 30 and about 600 mCi, more preferably between about 50 and less than about 500 mCi. When the antibody or ligand has a longer circulating half life relative to a foreign protein such as a murine antibody, an effective single treatment non-marrow ablative dosage of ¹³¹I labeled antibody ranges from between about 5 and about 40 mCi, more preferably less than about 30 mCi. Imaging dosages for a radioactive isotope label, e.g., the ¹¹¹In label, are typically less than about 5 mCi.

While ¹³¹I and ⁹⁰Y have been used extensively in the clinic, other radioactive isotopes are known in the art and can been used for similar purposes. Still other radioisotopes are used for imaging. For example, additional radioisotopes which can be used include, but are not limited to, ¹³¹I, ¹²⁵I, ¹²³I, ⁹⁰Y, ¹¹¹In, ¹⁰⁵Rh, ¹⁵³Sm, ¹⁶⁶Ho, ¹⁷⁷Lu, and ¹⁸⁸Re and ¹⁸⁶Re, ³²p, ⁵⁷Co, ⁶⁴Cu, ⁶⁷Cu, ⁷⁷Ga, ⁸¹Rb, ⁸¹Kr, ⁸⁷Kr, ¹¹³In, ¹²⁷Cs, ¹²⁹Cs, ¹³²I, ¹⁹⁷Hg, ²¹³Pb, ²¹⁶Bi, ¹¹⁷Lu, ²¹²Pb, ²¹²Bi, ⁴⁷Sc, ¹⁰⁵Rh, ¹⁰⁹Pd, ¹⁹⁹Au, ²²⁵Ac, ²¹¹At, and ²¹³Bi. In this respect alpha, gamma and beta emitters are all contemplated as aspects of the instant invention. Further, it is submitted that one skilled in the art could readily determine which radionuclides are compatible with a selected course of treatment without undue experimentation. To this end, additional radionuclides which have already been used in clinical diagnosis include ¹²⁵I, ¹²³I, ⁹⁹Tc, ⁴³K, ⁵²Fe, ⁶⁷Ga, ⁶⁸Ga, as well as ¹¹¹In. Antibodies have also been labeled with a variety of radionuclides for potential use in targeted immunotherapy, for example, as described in Peitersz et al. (1987) Immunol. Cell Biol. 65: 111-125. These radioactive isotopes include ¹⁸⁸Re and ¹⁸⁶Re as well as ¹⁹⁹Au and ⁶⁷Cu. U.S. Pat. No.5,460,785 provides information regarding such radioisotopes and is incorporated herein by reference.

Chemotherapeutic Agents:

The chemotherapeutic agents that can be used in the formulations and methods of the invention include taxanes, colchicine, vinca alkaloids, epipodophyllotoxins, camptothecins, antibiotics, platinum coordination complexes, alkylating agents, folic acid analogs, pyrimidine analogs, purine analogs or topoisomerase inhibitors. A preferred topoisomerase inhibitor is an epipodophyllotoxin. Preferred pyrimidine analogs include capecitabine, 5-fluoruracil, 5-fluorodeoxyuridine, 5-fluorodeoxyuridine monophosphate, cytosine arabinoside, 5-azacytidine, or 2′, 2′-difluorodeoxycytidine. Preferred purine analogs include mercaptopurine, azathioprene, thioguanine, pentostatin, erythrohydroxynonyladenine, cladribine, vidarabine, and fludarabine phosphate. Folic acid analogs include methotrexate, raltitrexed, lometrexol, permefrexed, edatrexate, and pemetrexed. A preferred epipodophyllotoxin is etoposide or teniposide. A preferred camptothecin is irinotocan, topotecan, or camptothecan. Preferably, the antibiotic is dactinomycin, daunorubicin (daunomycin, daunoxome), doxorubicin, idarubicin, epirubicin, valrubucin, mitoxanthrone, bleomycin, or mitomycin. A preferred platinum coordination complex is cisplatin, carboplatin, or oxaliplatin. Preferably, the alkylating agent is mechlorethamine, cyclophosphamide, ifosfamide, melphalan, dacarbazine, temozolomide, thiotepa, hexamethylmelamine, streptozocin, carmustine, busulfan, altretamine or chlorambucil.

Additional examples of chemotherapeutic agents can include alkylating agents such as thiotepa and cyclosphosphamide (CYTOXAN™);

alkyl sulfonates such as busulfan, improsulfan and piposulfan;

aziridines such as benzodopa, carboquone, meturedopa, and uredopa;

ethylenimines and methylamelamines including altretamine, triethylenemelamine, trietylenephosphoramide, triethylenethiophosphoramide and trimethylolomelamine;

acetogenins (especially bullatacin and bullatacinone);

camptothecins (including the synthetic analogue topotecan);

bryostatin; callystatin; CC-1065 (including its adozelesin, carzelesin and bizelesin synthetic analogues);

cryptophycins (particularly cryptophycin 1 and cryptophycin 8);

dolastatin; duocarmycin (including the synthetic analogues, KW-2189 and CBI-TMI);

eleutherobin; pancratistatin; sarcodictyin; spongistatin;

nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard;

nitrosureas such as carmustine, chlorozotocin, fotemustine, lomustine, nimustine, ranimustine;

antibiotics such as the enediyne antibiotics (e.g. calicheamicin, especially calicheamicin gamma1I and calicheamicin phiI1, see, e.g., Agnew (1994) Chem. Intl. Ed. Engl., 33:183-186; dynemicin, including dynemicin A; bisphosphonates, such as clodronate; esperamicin; as well as neocarzinostatin chromophore and related chromoprotein enediyne antibiotic chromomophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (Adriamycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin;

anti-metabolites such as methotrexate and 5-fluorouracil (5-FU);

folic acid analogues such as denopterin, methotrexate, pteropterin, trimetrexate;

folic acid replenisher such as folinic acid;

purine analogs such as fludarabine, 6-mercaptopurine, thiamiprine, thioguanine;

pyrimidine analogs such as ancitabine, azacitidine, 6-azauridine, carmofur, cytarabine, dideoxyuridine, doxifluridine, enocitabine, floxuridine;

androgens such as calusterone, dromostanolone propionate, epitiostanol, mepitiostane, testolactone;

anti-adrenals such as aminoglutethimide, mitotane, trilostane;

aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK®; razoxane; rhizoxin; sizofiran; spirogermanium; tenuazonic acid; triaziquone; 2, 2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; cytosine, arabinoside (“Ara—”);

cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine (Gemzar™); 6-thioguanine; mercaptopurine; methotrexate;

platinum analogs such as cisplatin and carboplatin;

vinblastine, vincristine; vinorelbine (Navelbine™);

etoposide (VP-16); ifosfamide; mitoxantrone;; novantrone; teniposide; edatrexate; daunomycin; aminopterin; xeloda; ibandronate; CPT-11;

topoisomerase inhibitor RFS 2000; difluoromethylornithine (DMFO);

retinoids such as retinoic acid; capecitabine; and pharmaceutically acceptable salts, acids or derivatives of any of the above.

Additional preferred chemotherapeutic agents include those used in combination therapies, for example, CHOP, and so forth. In particular embodiments, such combination therapies can be used with the anti-CDIM binding antibodies, or in combination with additional cytotoxic antibodies, in particular anti-CD22, anti-CD52 and anti-CD20 antibodies.

Particularly preferred are agents that arrest the B cell in its cell cycle, such as agents that interfere with the polymerization or depolymerization of microtubules. Exemplary agents include colchicine, the vinca alkaloids, such as vincristine, vinblastine, vindesine, or vinorelbine, and taxanes, such as taxol, paclitaxel, and docetaxel. Additional preferred agents are anti-actin agents. In a preferred embodiment, the anti-actin agent is jasplakinolide or cytochalasin, which can be used more preferably in an ex vivo method, such as a method of purging bone marrow of malignant cells. Mixtures of any of the above agents can also be used, such as CHOP, CAMP, DHAP, EPIC, and the like, as discussed in U.S. Patent Application No. 2004/0136951, incorporated by reference herein.

Toxins

Toxins can be administered as immunoconjugates, ligand conjugates, or co-administered with an antibody. Toxins include, without limitation, Pseudomonas exotoxin A, ricin, diphtheria toxin, momordin, pokeweed antiviral protein, Staphylococcal enterotoxin A, gelonin, maytansinoids (e.g., as described in U.S. Pat. Nos. 6,441,163), or the like.

Cell Growth Regulators and/or Inhibitors

Cell growth regulators and/or inhibitors include small molecule therapeutics such as hormones or anti-hormonal agents, kinase inhibitors, proteasome inhibitors, gene therapy agents or gene expression modifiers.

Anti-hormonal agents can be useful particularly in the therapy of autoimmune diseases where hormonal exacerbation is implicated, particularly estrogenic action in women. Anti-hormonal agents act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); aromatase inhibitors that inhibit the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprolide, and goserelin; and pharmaceutically acceptable salts, acids or derivatives of any of the above. Androgenic hormones can be especially useful in the treatment of autoimmune disease, and a representative androgenic hormone is dihydroepiandrosterone (DHEA). Selective androgen receptor modulators (SARMs) include for example, the compounds described in U.S. Pat. No. 6,645,974 to Hutchinson, such as androstane and androstene carboxamides.

Kinase inhibitors are widely known, and particularly preferred kinase inhibitors include the bcr/abl tyrosine kinase inhibitors, such as imatinib (Gleevec) and its related compounds, as described in U.S. Pat. No. 5,521,184 to Zimmermann. Additional tyrosine kinase inhibitors can include agents that block signaling complexes involved in the activation of and transcription of Lyn kinase, including for example, siRNAs that blocks the activity of Lyn kinase. Yet additional kinase inhibitors include compounds such as AGL 2592 described in Ben-Bassat, H. et al., (2002) J. Phannacol. Exp. Ther. 303:163 shown to be apoptosis inducing for non-Hodgkin lymphomas; herbimycin A as described by Mahon, T M and O'Neill, L A (1995) J. Biol. Chem. 270:28557 shown to block DNA binding and NF-kappa B-driven gene expression; indolinone compounds such as those described in U.S. Patent No. 6,680,335 to Tang; pyrazolopyrimidine derivatives such as those described in U.S. Patent No. 6,660,744 to Hirst, and the like. Proteasome inhibitors include the boronic esters described in U.S. Pat. No. 6,083,903 to Adams. A preferred proteasome inhibitor is bortezomib (Velcade).

Gene therapy agents and gene expression modifiers include antisense nucleic acid sequences, interfering nucleic acid sequences and the like. The gene therapy agents and gene expression modifiers can be used either as an immunoconjugate or as a separately administered cytotoxic agent. Particularly useful gene therapy agents and gene expression modifiers include those that encode proteins involved in pro-apoptotic pathways, as well as those that block inhibitors of the pro-apoptotic pathways or those that block proliferative signaling, all of which can contribute to uncontrolled growth and hyperproliferation. For example, gene expression modifiers can include antisense or siRNA that act to inhibit the NF-kB pathway, thereby inhibiting the abnormal proliferation present when this pathway is abnormally activated.

Antisense DNA oligonucleotides are typically composed of sequences complementary to the target sequence, usually a messenger RNA (mRNA) or an mRNA precursor. The mRNA contains genetic information in the functional, or sense, orientation and binding of the antisense oligonucleotide inactivates the intended MRNA and prevents its translation into protein. Such antisense molecules are determined based on biochemical experiments showing that proteins are translated from specific RNAs and once the sequence of the RNA is known, an antisense molecule that will bind to it through complementary Watson-Crick base pairs can be designed. Such antisense molecules typically contain between 10-30 base pairs, more preferably between 10-25, and most preferably between 15-20. The antisense oligonucleotide can be modified for improved resistance to nuclease hydrolysis, and such analogues include phosphorothioate, methylphosphonate, phosphoroselenoate, phosphodiester and p-ethoxy oligonucleotides as described in WO 97/07784.

The gene therapy agent can also be a ribozyme, DNAzyme, catalytic RNA, or a small interfering RNA (siRNA). RNA interference utilizes short RNAs typically less than about 30 base pairs, which act through complementary base pairing as described above. The siRNAs can be linear or circular.

As mentioned above, agents and modifiers that block signaling complexes involved in the activation of and transcription of Lyn kinase, would be advantageous. In a particular embodiment, an siRNA that blocks the activity of Lyn kinase, such as the siRNA reported by Ptasznik, A et al., (2004) Nat. Med.10:1187, can be administered with the anti-CDIM binding antibody either as an immunoconjugate or as a separately administered cytotoxic agent.

Pharmaceutical Formulations

Antibodies and cytotoxic agents can be formulated using any methods and pharmaceutically acceptable excipients known in the art. Typically, antibodies are provided in saline, with optional excipients and stabilizers. Chemotherapeutic agents can vary widely in formulation methods and excipients, and this information is available for example, in Remington's Pharmaceutical Sciences (Arthur Osol, Editor).

Cytotoxic Antibodies:

Cytotoxic antibodies that are useful in the present invention include antibodies having specific binding for any cell surface molecule on a B cell. Cell surface molecules include receptors, immunoglobulins, cytokines, glycoproteins, etc. For example, the cytotoxic antibody can exhibit specific binding for CD11a, CD19, CD20, CD21, CD22, CD25, CD34, CD37, CD38, CD40, CD45, CD52, CD80, CD 86, IL-4R, IL-6R, IL-8R, IL-13, IL-13R, α-4/β-1 integrin (VLA4), BLYS receptor, cell surface idiotypic Ig, tumor necrosis factor (TNF),or mixtures thereof, without limitation. For example, the cytotoxic antibody having specific binding for CD11a can be, for example, efalizumab (RAPTIVA). The cytotoxic antibody having specific binding for CD20 can be rituximab (RITUXAN). The cytotoxic antibody having specific binding for CD22 can be, for example, epratuzumab. The cytotoxic antibody having specific binding for CD25 can be, for example, daclizumab (ZENAPAX) or basiliximab (SIMULECT). Antibodies to CD52 include, e.g., CAMPATH. Antibodies to α-4/β-1 integrin (VLA4) include, e.g., natalizumab. Antibodies to TNF include, for example, infliximab (REMICADE).

The cytotoxic antibodies can be used as part of a combined immunotherapy regimen for treatment of autoimmune disease, lymphoid cancer, and other B cell hyperproliferative diseases associated with viral diseases and immunodeficiencies. Thus in preferred embodiments, the antibody having specific binding for CDIM epitopes on a B cell can be used in a combined immunotherapy regimen with epratuzumab, RITUXAN, ZENAPAX, REMICADE or RAPTIVA, for example, or in combinations thereof.

The cytotoxic antibody can also be used as an immunoconjugate comprising a radioactive isotope or toxin, for example. Further, in additional embodiments, a combined therapy can be used comprising the antibody having specific binding for CDIM epitopes on a B cell, an additional cytotoxic antibody having specific binding for cell surface molecules on a B cell, and one or more chemotherapeutic agents. For example, mAb216 could be used in combination with an anti-CD20 antibody such as rituximab, tosutimab, or ibritumomab, in combination with anti-CD22, for example, epratuzumab, or in combination with an anti-CD52 antibody such as CAMPATH. The combination therapy can further include chemotherapy, such as an agent that disrupts the cytoskeleton of the cell, e.g., vincristine, in a combined chemotherapy and immunotherapy regimen.

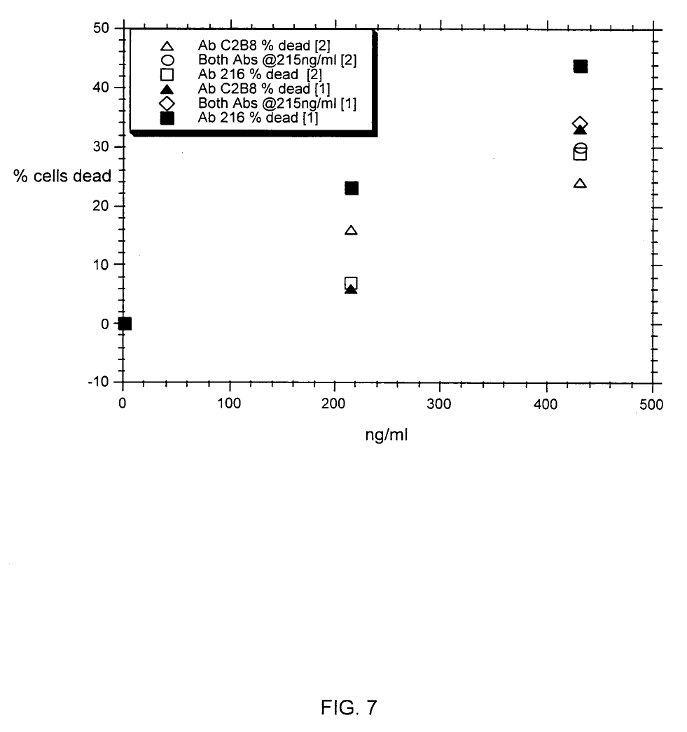

The combination of CDIM binding antibodies such as VH4-34 antibodies with cytotoxic antibodies directed against different cell surface antigens is efficacious, as discussed in Example 10 and shown in FIG. 7, providing a result that is at least additive, and in some instances could be synergistic. Further, as shown in FIG. 4, mAb 216 is highly effective in killing many of the cells obtained from patients with relapsed or refractory B cell lymphoma.

The combination of mAb 216 or other VH4-34 antibody directed against the CDIM epitope is expected to combat the incidence of Rituxan resistant cells, and increase the efficacy of Rituxan treatment as well as increase the efficacy of mAb 216 treatment.

Particular B cell antigens include B lymphocyte stimulator (BLyS) is a member of the tumor necrosis factor (“TNF”) superfamily that induces both in vivo and in vitro B cell proliferation and differentiation (Moore et al., Science 285: 260-263 (1999)). Levels of BLyS protein have been found to be elevated in patients with autoimmune disease, including systemic lupus erythematosus (SLE), rheumatoid arthritis, and Sjogren's syndrome (Zhang et al., The Journal of Immunology, (2001) 166:6-10; Cheema et al., Arthritis and Rheumatism (2001) 44:1313-1319; and Groom et al., Journal of Clinical Investigation (2002) 109:59-68). Administration of a soluble form of a BLyS receptor, TACI, has been shown to alleviate the autoimmune phenotype of NZBWF1 and MRL-lpr/lpr mice (Gross et al., Nature, (2000) 404:995-999). Thus, antibodies and related molecules that bind to BLyS may find medical utility in the treatment of B cell associated diseases and disorders, including autoimmune disorders and lymphoid cancers.

Cell surface idiotypic Ig is a patient specific marker present on lymphoid cancers of B cell origin. These cell surface receptors also provide a useful target for cytotoxic antibody therapies, and are useful in the methods described herein. Preparation of anti-idiotope antibodies to these patient specific cell surface Igs is described in U.S. Pat. No. 5,972,334 to Denney.

Modes of Administration

The antibodies of the invention may be administered to the human or animal patient by a variety of different means, typically via parenteral administration, Any other means of administration found to be effective for administering antibodies and cytotoxic agents in functional form can be utilized, for example, orally, topically, or via an implanted reservoir. Topical administration includes passive or active means, e.g., using a patch, a carrier, or iontophoresis; transmucosal, e.g., sublingual, buccal, rectal, vaginal, nasal, or transurethral, topical delivery to the lung, bronchi and nasal passages, e.g., via inhalation of nebulized of powdered active agent. Oral administration includes generally gastric or duodenal. Parenteral injection includes injection into a body cavity or vessel, e.g., intraperitoneal, intravenous, intralymphatic, intratumoral, intramuscular, interstitial, intraarterial, subcutaneous, intralesional, intraocular, intrasynovial, or intraarticular, intrasternal, intracerebrovascular (e.g., intracerebral, intraventricular, intrathecal), intrahepatic, intralesional and intracranial injection or infusion techniques. Preferably, the compositions are administered orally or intravenously.

It is to be understood that while the invention has been described in conjunction with the preferred specific embodiments thereof, that the description above as well as the examples that follow are intended to illustrate and not limit the scope of the invention. The practice of the present invention will employ, unless otherwise indicated, conventional techniques of preparing pharmaceutical formulations and the like, which are within the skill of the art. Other aspects, advantages and modifications within the scope of the invention will be apparent to those skilled in the art to which the invention pertains. Such techniques are explained fully in the literature.

All patents, patent applications, and publications mentioned herein, both supra and infra, are hereby incorporated by reference.

In the following examples, efforts have been made to ensure accuracy with respect to numbers used (e.g., amounts, temperature, etc.) but some experimental error and deviation should be accounted for. Unless indicated otherwise, temperature is in degrees ° C. and pressure is at or near atmospheric.

Abbreviations:

ALL Acute Lymphocytic Leukemia

NHL Non-Hodgkins Lymphoma

CLL Chronic Lymphocytic Leukemia

VCR vincristine

IV intravenous

IP intraperitoneal

Ig immunoglobulin

### EXAMPLE 1

### mAb 216 Binds to CD19+ Bone Marrow Cells From Tumor Cell Bank

Twenty-seven fully characterized bone marrow samples were obtained from the Children's Oncology Group tumor cell bank. Thawed cells were stained with biotinylated mAb 216 and fluorescent-labeled streptavidin. MAb 216 bound to all 15 samples of CD19+ B-progenitor ALL with a mean channel fluorescence (MCF) of 717 (range 225-1020). Twelve samples of T cell ALL were tested and showed a MCF of 62 (range 28-149); 3 T-ALL had mAb 216-binding above background. The results are shown in FIG. 1, with the relative binding intensities indicated by +,” “++” and “+++,” and nonbinding indicated by “−”. As indicated in FIG. 1, mAb 216 binds to B cell lymphomas and leukemias of all types; but does not demonstrate significant binding to T cell lymphoma.

### EXAMPLE 2

### mAB 216 Kills Pre-B ALL Cells from Bone Marrow

Twelve specimens of fresh bone marrow (BM) were obtained from patients undergoing diagnostic bone marrow aspiration for leukemia and were analyzed in vitro for mAb 216-binding and cytotoxicity at 24 hours. Immunophenotyping for expression of CD19, CD10, CD34, CD20, CD3, CD2, and binding of biotin-labeled mAb 216 was performed on all samples. Cytotoxicity was assayed by washing and incubating BM overnight with 20 μg/ml mAb 216 or control IgM. Incubated cells were stained with FITC anti-CD19 and propidium iodide (PI). Cell death was measured by a change in % CD19+ cells and by PI uptake in CD19-expressing cells by flow cytometry.

Cytotoxicity, measured as percent of cells killed following incubation with mAb 216 as compared to incubation with control IgM, was as follows for BM from patients with pre-B ALL: 60-90% (n=4), 30-50% (n=4), and 7-20% (n=2). Increased cytotoxicity correlated with intensity of mAb 216 binding by MCF, and may be related to cell cycle-dependent differential expression of the ligand for mAb 216. BM samples from patients with T-ALL (n=1) and AML (n=1) were not killed by mAb 216.

### EXAMPLE 3

### Antibodies Kill B Cells in an Animal Model of B Cell Leukemia

Experiments with the human pre B cell Nalm-6 model of B cell leukemia in CB17 SCID and NOD/LtSz-SCID immunodeficient mice have shown increased survival and a 20% cure rate following treatment with mAb 216 ²². Nalm-6 is a cell line derived from ALL that does not express the mature B cell antigen CD20 and gives a reproducible intravenous model of human tumor in the. SCID mouse ²³. To treat mice, purified mAb 216, (400 μgs/200 μl) was injected intravenously (IV) on days 1, 7, 14, and 21 post engraftment. Comparing mice to humans in body surface area, the mice received the human equivalent of 90-100 mg/m² with each dose. Mice were observed for a period of 100 days for tumor development.

One mg of purified mAb 216 (human equivalent of approximately 220-250 mg/m²) and control polyclonal human IgM was injected IV in four Balb/c mice. At 24 hours blood was collected. A chemistry panel, which included creatinine, bilirubin, alkaline phosphatase, SGOT (AST), and SGPT (ALT) showed some slight liver enzyme elevations but with normal bilirubin. At 14 days all values except alkaline phosphatase, in both controls and test mice, had returned to normal. Mice were alive and well at 8 weeks. Balb/c mice were given 500 μgs of mAb 216 IV and sacrificed at 24 and 48 hr post injection. Histology of spleen, kidney, liver, and heart showed no pathology. CB17-SCID/SCID mice were also injected with mAb 216 (200 μgs/injection, IP or IV) on days 0, 3 and 10. All mice, regardless of the mode of mAb injection (IV or IP) were in apparent good health 6 weeks post last injection.

### EXAMPLE 4

### mAb 216 Wounds B Cell Membranes and Invokes a Resealing Response by Lysosomes

The natural response to membrane damage is rapid resealing by addition of internal lysosomal membrane at the wound site. Lamp-1 is an abundant lysosomal membrane glycoprotein normally not present on the plasma membrane (Granger, B. L., et al. (1990) J. Biol. Chem. 265:12036; McNeil, P. L. (2002) J. Cell Sci. 115:873). When lysosomes are induced to fuse with plasma membrane, the intra-lysosomal NH₂-terminal domain of Lamp-i becomes exposed on the cell surface. This fusion event can be monitored by surface staining of live cells with mAbs directed to the lumenal epitope of Lamp-1 (Reddy, A., et al. (2001) Cell 106:157; Rodriguez, A., et al. (1997) J. Cell. Biol. 137:93; Martinez, I., et al. (2000) J. Cell. Biol. 148:1141). Thus, the presence of Lamp-1 on the cell surface is an indication of membrane resealing following membrane disruption (McNeil, P. L., and R. A. Steinhardt (2003) Ann. Rev. Cell Dev. Biol. 19:697).

To test whether the VH4-34 encoded mAb 216 wounds cells and thus invokes a rapid repair and resealing response, human B cell lines treated with mAb 216 were assayed for the swift appearance on the cell surface of the lysosome-specific protein Lamp-1.

Cells and Reagents

Human Pre-B cell line Nalm-6 (Hurwitz, R., et al. (1979) Int. J. Cancer 23:174), Reh (Rosenfield, C., A. et al. (1977) Nature 267:841), and mature B-cell line OCI-Ly8 Tweeddale, M. E., et al. (1987) Blood 69:1307) were maintained in logarithmic phase in Iscove's medium with heat inactivated 10% FCS. B cell lines were obtained from ATCC. VH4-34 encoded mAbs, mAb 216 (Bhat, N. M., et al. (1993) J. Immunol. 151:5011), Z2D2 (Bhat, N. M., et al. (2000) Scand. J. Immunol. 51:134), and Y2K as well as isotype-matched control mAb, MS2B6, derived from a member of the VH3 family (Glasky, M. S., et al. (1992) Hum. Antibod. Hybridomas 3:114), were produced in the laboratory and purified from serum free hybridoma supernatant by 2× precipitation with water. MAbs were concentrated when necessary on a Centriprep concentrator (Amnicon, Dancers, Mass.). Purity of the IgM mAbs, checked by polyacrylamide gel electrophoresis, was 90-95% pure. Concentration of purified IgMs was determined by sandwich ELISA using human IgM as a standard (catalog # 31146, Pierce Biochemicals, Rockford, Ill.). In addition to MS2B6, the Pierce IgM was also used as an isotype control. All mAbs were sterile-filtered and free of sodium azide.

Cell Viability Assay Using PI Staining and Forward Scatter

The integrity of the plasma membrane was assessed by the ability of cells to exclude propidium iodide (PI, Sigma, St. Louis, Mo.). The level of PI incorporation was quantitated by flow cytometry on FACScan (Becton-Dickinson, San Jose Calif.) interfaced with VersatermPro and FlowJo software at Stanford's FACS facility. PI-negative cells with normal size as measured by forward scatter signals were considered live cells.

Briefly, cells were treated as specified in each experiment and resuspended in PBS with 3% FCS and 10 μgs/ml of PI. In experiments where toxicity was evaluated in Ca-free medium, cells were resuspended in appropriate media with or without calcium to which 10 μgs/ml PI was added. Since previous studies have shown that mAb 216-mediated toxicity is remarkably pronounced at lower temperatures (Bhat, N. M., et al. (1996) Clin. Exp. Immunol. 105:183), precautions were taken to keep all media and cells at 37° C. and the centrifuge at room temperature.

ATP Depletion and Release Assay

Intracellular and released ATP was measured according to manufacturer's instructions by the bioluminescence assay kit (Catalog # A-22066, Molecular Probes). Standard ATP dilutions ranging from 1 nM to 1 μM were tested as positive control. Cells were exposed to various concentrations of mAb 216, in different media as specified in each experiment. 10 μl of reaction supernatant was added to 90 μl of the standard reaction solution that contained DTT, luciferin and luciferase. Light generation, in the presence of ATP as a cosubstrate, was immediately measured by luminometer (Lumimark Microplate Reader, Bio-Rad) interfaced with MicroWin 2000, version 4.2 software (Mikrotek Laborsysteme, Gmbh). This assay allows detection of femtomolar quantities of ATP. To assess the intracellular ATP content, cells were lysed with 1% NP-40 at RT for 10 minutes, and 10 μl of the lysate was tested as described above.

Lamp-1 Expression Studies

Surface Lamp-1 expression was studied by epi-fluorescence, flow cytometry and confocal microscopy. Antibodies to the lumenal epitope of human Lamp-1 (CD107a, clone H4A3) and the isotype control for Lamp-1, a mouse IgGIk were obtained from BD-PharMingen. Both antibodies were detected with a secondary FITC-conjugated Goat F(ab)₂ anti-mouse IgG (Pierce Biochemicals). Cells (5×10⁵) were exposed to various concentrations of mAb 216 or human IgM control (mAb MS2B6 or Pierce IgM) for the specified time in each experiment at 37° C. Cells were then fixed with 2% pre-warmed paraformaldehyde at RT for 20 minutes, washed twice with pre-warmed media and stained with anti-Lamp-1 or isotype control for 15 minutes. Cells were then washed twice with staining medium (PBS with 3% FCS and 0.2% sodium azide) and incubated with secondary antibody to anti-Lamp-1 for another 15 minutes. After two washings, cells were resuspended in staining medium and analyzed by flow cytometry, immunofluorescence or confocal microscopy.

Confocal imaging was performed at Stanford's Cell Sciences Imaging Facility on the MultiProbe 2010 laser confocal microscope (Molecular Dynamics, Sunnyvale, Calif.). The MultiProbe uses an Ar/Kr mixed gas laser with excitation lines of 488, 568 and 647 and is built on a Nikon Diaphot 200 inverted microscope. With an excitation wavelength of 488 nm, the emitted light was passed through a 510LP beamsplitter and collected with a 510 long pass filter. A Nikon 60X (NA 1.4) planapo objective was used. Epi-fluorescence imaging was performed on Axioplan 2 Microscope (Carl Zeiss, Inc., GmbH) equipped with AxioCam HRc camera (Carl Zeiss) and Opti-Quip Power Supply (Model 1200, Highland Mills, New York) interfaced with Axiovision 3.1 software (Carl Zeiss). Flow cytometry was performed on FACScan.

Results and Conclusions

Lamp-1 expression on untreated cells varied from as low as 5% to 50% from experiment to experiment. The variation occurs due to standard laboratory handling of B cell lines. In experiments where baseline level of lamp-1 expression was 50%, isotype control treated cells remained 50% positive and mAb 216 treated cells were 100% Lamp-1 positive. Lamp-1 staining on cell lines was repeated 5 times to ensure reproducibility. Results are discussed from experiments where baseline Lamp-1 expression is 5%.

Nalm-6 cells exposed to mAb 216 for 1 minute demonstrated a dramatic increase in Lamp-1 staining, but cells exposed to isotype control or cells with no treatment did not increase their Lamp-1 expression. Lamp-1 exposure was also observed in other B cell lines, OCI-Ly8 (mature-B) and Reh by FACS and epi-fluorescence (data not shown). Membrane integrity of cells was simultaneously assessed for each sample by PI uptake. Cells remained PI negative at 1 minute post 216 exposure.

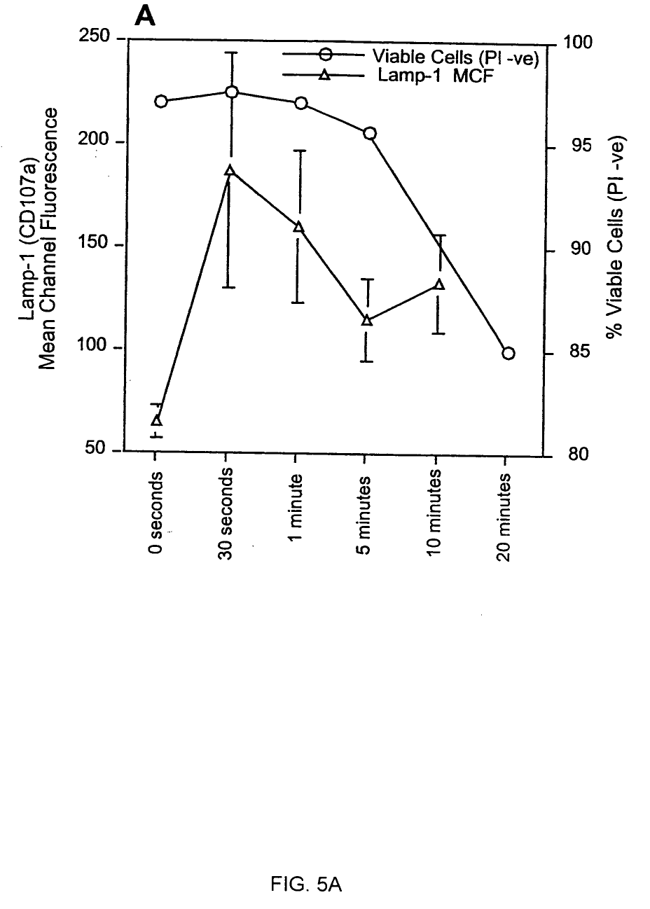

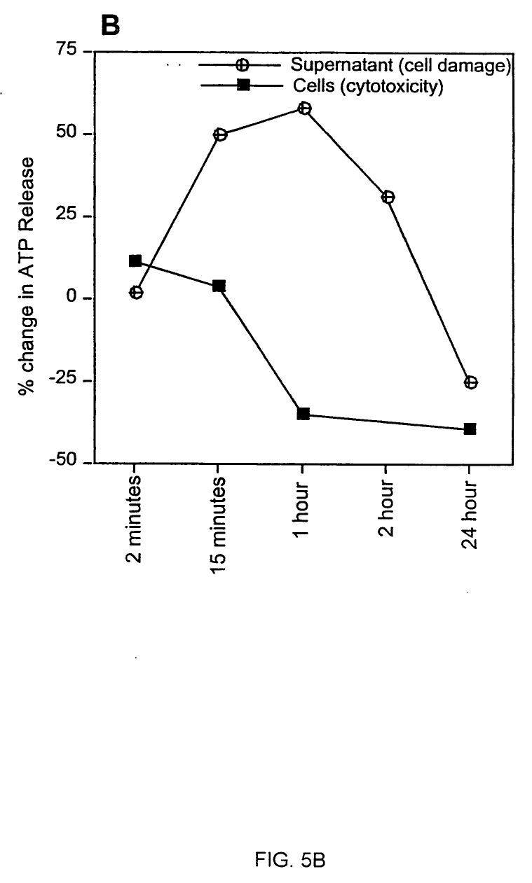

Lamp-1 staining and PI uptake was also measured at different time points post mAb 216 exposure. Lamp-1 exposure was a rapid event with the brightest staining observed at 30 seconds of Ab exposure, dropping gradually in the next 5 minutes (FIG. 5A). Cells remained PI-negative during this time period. PI uptake was demonstrated after about 5 minutes of exposure to mAb 216, and by 20 minutes, 10-25% of cells became membrane permeable, as evinced by PI uptake.

Membrane disruption measured by release of ATP also showed a similar time course. As shown in FIG. 5B, ATP was not detected in the supernatant at 2 minutes, a time-point where Lamp-1 is detected on the cell membrane. But at 15 minutes and 1 hr ATP release increased, suggesting membrane damage occurred that could not be resealed. At 2 and 24 hr post mAb 216-treatment, there was a decrease in measured ATP that may be the result of cell lysis and necrosis that degrades the released ATP. When ATP content in the cell pellet is evaluated, the bioluminescent assay becomes a measure of cell proliferation and cytotoxicity. The cytotoxic effects of mAb 216 were apparent within 1 hr of exposure.

These results demonstrate that mAb 216 mediated membrane damage is repaired by the same mechanism that restores cell viability after injury by mechanical or physical wounding, indicating that mAb 216 treatment results in a cell wounding event similar to any other large membrane disruption. Cell wounding by an antibody has not heretofore been observed. The membrane damage by mAb 216 was initially resealed as internal membrane was added rapidly to the lipid bilayer, but with increased time of exposure to mAb 216, attempts to reseal failed and the membrane became permeable to both PI and ATP. In addition to mAb 216, other anti-B-cell VH4-34 encoded IgM mAbs mediated similar membrane damage and invoked a similar resealing response by lysosomes.

### EXAMPLE 5

### Repair of mAb 216 Induced Membrane Damage is Dependent on Functional Actin

As discussed by McNeil, P. ((2002) J. Cell Sci. 115:873) and others, membrane wound repair involves actin dependent processes. To test whether repair of membrane wounding induced by mAb 216 utilizes actin dependent repair mechanisms, cells were treated with agents that affect actin polymerization, and the effect on the repair of the membrane wound induced by mAb 216 was assessed. Cells were treated with cytochalasin or jasplakinolide, two agents that have opposite effects on actin polymerization. Cytochalasin depolymerizes actin into monomers, whereas jasplakinolide, a cyclic peptide obtained from a marine sponge, immobilizes actin in its filamentous form. Both treatments hinder actin-based cytoskeletal activities.

Methods:

Cytochalasin was obtained from Sigma and jasplakinolide was obtained from Molecular Probes (Eugene, Oreg.). Caspase inhibitors, Ac-IETD-CHO and Ac-DEVD-CHO were obtained from PharMingen (San Diego, Calif.). Nalm-6 cells (1×10⁶ cells/ml) were treated with jasplakinolide (3 μgs/ml), cytochalasin (5 μgs/ml), or caspase inhibitors (10 μM) for 2 hr at 37° C. before treatment with mAb 216. Control samples with equivalent amounts of DMSO were set in parallel. Cells were then exposed to 25 μg of mAb 216 or control Ab and analyzed by flow cytometry.

Results:

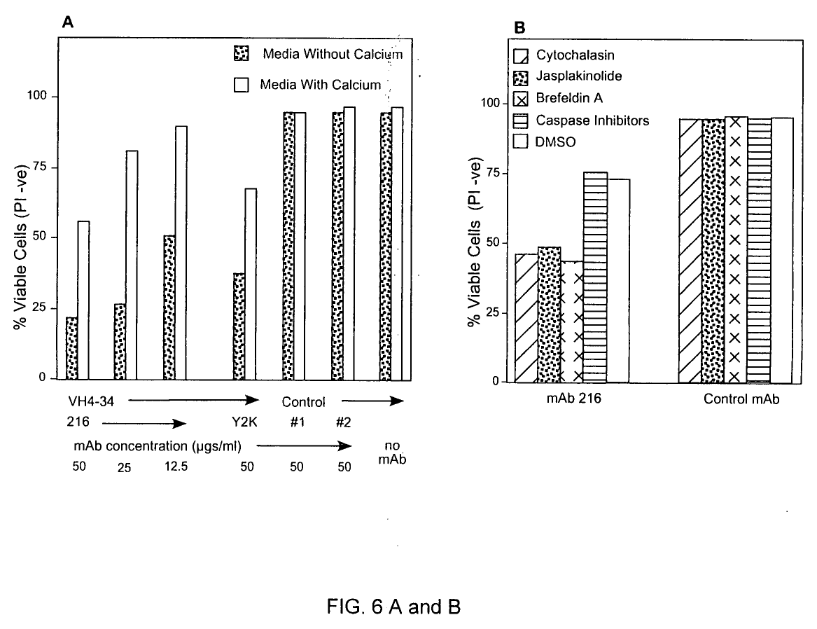

Cells treated with cytochalasin or jasplakinolide and mAb 216 showed decreased viability (percent viable cells) and hence increased susceptibility to mAb 216, demonstrating a synergistic effect and indicating a requirement for functional actin in the repair process. Cells treated with cytochalasin or jasplakinolide and control antibodies did not show a decrease in viability. Data from one representative experiment is shown in FIG. 6B. Similar results were obtained from three other experiments.

Incubation of cells with the caspase inhibitors Ac-IETD-CHO and Ac-DEVD-CHO did not alter cell viability, indicating that the mechanism of cell death is not due to apoptosis.

These results further support the mechanism of antibody induced cell membrane wounding caused by exposure to these antibodies.

### EXAMPLE 6

### Repair of mAb 216 Induced Membrane Damage is Dependent on Calcium

Since exocytosis of lysosomes is known to be a calcium dependent phenomenon (Miyake, K., and P. L. McNeil (1995) J. Cell Biol. 131:1737; Bi, G. Q., et al. (1995) J. Cell Biol. 131:1747), membrane wounding by mAb 216 nd repair of the wound was tested in calcium free and normal calcium conditions. The cell viability of Nalm-6 cells when treated with two VH4-34 encoded mAbs, mAb 216 at 50,25 and 12.5 ng/ml concentrations, and Y2K at 50 ng/ml, was tested in the presence of media with and without calcium. As shown in FIG. 6A, cell viability decreased significantly in the absence of calcium, indicating that calcium was necessary for the wound repair. Cells treated with control antibodies or no antibody did not show any change in cell viability in the presence or absence of calcium. Other B cell lines, OCI-Ly8 and Reh also showed a similar increase in cytotoxicity in calcium-free conditions (data not shown).

### EXAMPLE 7

### Repair of mAb 216 Induced Membrane Damage is Dependent on Functional Golgi

Treatment with Brefeldin A (BFA) is known to result in release of golgi-associated coat proteins, redistribution of the golgi membrane into the endoplasmic reticulum and a block in secretion from golgi apparatus (Klausner, R. D., (1992) J. Cell Biol. 116:1071). Newly formed lysosomes are not generated in BFA treated cells, thus providing a condition to test their requirement in wound repair. Therefore, the ability of newly formed lysosomes to aid in the repair of the membrane wounds induced by mAb 216 cells was tested by treating cells with BFA.

Methods:

Brefeldin-A was obtained from Sigma. Nalm-6 cells (1×10⁶ cells/ml) were treated with BFA (25 μg/ml) for 2 hr at 37° C. before treatment with mAb 216. Control samples with equivalent amounts of DMSO were set in parallel. Cells were then exposed to 25 μg of mAb 216 or control Ab and analyzed by flow cytometry.

Results:

As shown in FIG. 6B, the cell viability (percent viable cells) was decreased by the combination of BFA and mAb 216, demonstrating a synergistic effect on viability. BFA had no effect on the viability of cells treated with control antibodies. This result demonstrates that membrane repair was blocked by BFA, suggesting that newly generated lysosomes are necessary for membrane repair and the continued survival and integrity of mAb 216-wounded B-cell lines. This result thus further confirms that mAb 216 generates membrane wounds on B cells, and that the cells attempt to patch the wound utilizing lysosomal fusion with the plasma membrane. When the generation of additional lysosomes is inhibited by BFA, the repair process may not be adequate to maintain cell viability.

### EXAMPLE 8

### Synergistic B Cell Killing with Vincristine

Enhanced cell killing was demonstrated when mAb 216 was combined with chemotherapeutic agents, particularly with vincristine, in cytotoxicity assays directed against B cell lines. Three cell lines which have been derived from ALL blasts of different genotype and phenotype, Nalm 6, REH, and SUPB15, were incubated with mAb 216 alone or in combination with vincristine (VCR), for 48 hours at 37° C.

As shown in FIG. 4, and Table 1 below, these results show that at low vincristine concentrations (0.2 ng/ml), no cell death occurred due to treatment with vincristine alone. However, when vincristine was combined with mAb 216, the percentage of B cells killed more than doubled, demonstrating a synergistic interaction. The cytotoxicity of mAb 216 for B-progenitor lymphoblasts, alone and in combination with chemotherapy, makes this antibody a promising reagent for further immunotherapy studies in childhood ALL.

### EXAMPLE 9

### Enhanced Cytotoxicity of mAB 216 to B Cell Lines by Chemotherapeutic Agents

In vitro cytotoxicity of mAb216 in combination with single chemotherapeutic agents was tested. Three cell lines which have been derived from ALL blasts of different genotype and phenotype, Nalm 6, REH, and SUPB15, were incubated with mAb 216 alone or in combination with vincristine (VCR), daunorubicin (DNR), or L-asparaginase (ASPR). All of the chemotherapeutic agents when used in combination with mAb 216 resulted in a greater degree of cytoxicity than was seen with either single agent chemotherapy or mAb 216 alone. However, the combination of vincristine with mAb 216 was most efficacious, resulting in a magnitude of cytoxicity that was synergistic compared to the amount of cell killing induced by either vincristine or mAb 216 alone. These results demonstrate enhanced cytotoxicity of mAb 216 in the presence of chemotherapeutic agents, in part at least because mAb 216 treatment results in permeabilization of the B cells and allows otherwise impermeable chemotherapeutic agents access to the cell interior.

The results are presented in Table 1 below.

TABLE 1
In vitro cytotoxicity of mAb216 in combination with
chemotherapeutic agents

| Cell line/ | | | % change in |
|---|---|---|---|
| Incubation time | Treatment | Live cells × 10⁵ | live cells |
| Nalm 6 48 h | control | 13 | |
| | mAb 216 5 μg/ml | 10 | 23 |
| | VCR 0.2 ng/ml | 13 | 0 |
| | mAb 216 + VCR | 6 | 53 |
| Nalm 6 48 h | control | 8.2 | |
| | mAb 216 5 μg/ml | 5.6 | 31 |
| | DNR 5 ng/ml | 4.3 | 47 |
| | mAb 216 + DNR | 1.5 | 81 |
| Nalm 6 48 h | control | 11 | |
| | mAb 216 5 μg/ml | 7.1 | 35 |
| | VCR 2 ng/ml | 5 | 54 |
| | DNR 5 ng/ml | 4.5 | 59 |
| | mAb 216 + VCR | 0.28 | 97 |
| | mAb 216 + DNR | 4.3 | 61 |
| Nalm 6 48 h | control | 12 | |
| | mAb 216 5 μg/ml | 5.1 | 57 |
| | ASPR 0.8 U/ml | 9.2 | 23 |
| | mAb 216 + ASPR | 3.2 | 73 |
| REH 48 h | control | 8.6 | |
| | mAb 216 5 μg/ml | 4.6 | 46 |
| | VCR 2 ng/ml | 4.2 | 86 |
| | mAb 216 + VCR | 0.45 | 94 |
| REH 48 h | control | 13 | |
| | mAb 216 5 μg/ml | 11 | 15 |
| | VCR 2 ng/ml | 7.7 | 40 |
| | DNR 5 ng/ml | 4.5 | 65 |
| | mAb 216 + VCR | 0.9 | 93 |
| | mAb 216 + DNR | 4.1 | 68 |
| REH 48 h | control | 9.6 | |
| | mAb 216 5 μg/ml | 3.4 | 65 |
| | ASPR 0.8 U/ml | 6.2 | 35 |
| | mAb 216 + aspar. | 2.4 | 75 |
| SUP B15 48 h | control | 5.1 | |
| | mAb 216 5 μg/ml | 3.6 | 29 |
| | VCR 2 ng/ml | 2.8 | 45 |
| | DNR 4 ng/ml | 0.44 | 91 |
| | mAb 216 + VCR | 1.5 | 50 |
| | mAb 216 + DNR | 0.38 | 92 |
| SUP B15 48 h | control | 5.7 | |
| | mAb 216 5 μg/ml | 4.3 | 24 |
| | ASPR 0.8 U/ml | 3 | 47 |
| | mAb 216 + ASPR | 2.3 | 60 |

VCR; vincristine,
DNR; Daunorubicin,
ASPR; asparginase

### EXAMPLE 10

### Combined Treatment of mAb 216 and C2B8 (Anti-CD20 Ab)

To investigate whether mAb216 and an anti-CD20 antibody could provide an efficacious combination in vivo, the effect of the combined antibody treatment on B cells was tested in the presence of complement, as would be encountered during in vivo treatment in a human patient.

The lymphoma cell line OCI-Ly8 was treated with mAb 216 or C2B8 (Rituxan®) in the presence of rabbit complement. Cytotoxicity was detected using the MTT assay, which measures the colorimetric change of 3(4,5)-dimethylthiazol-2,5-diphenyl tetrazolium bromide, a measure of function of mitochondrial enzymes, to determine the % cells killed. Cells were plated at densities of 1×10⁵ per ml or 3×10⁵ per ml. Each antibody was tested separately at 215 ng/ml or 430 ng/ml, and the combined treatment consisted of each antibody at 215 ng/ml for a combined concentration of 430 ng/ml. The results shown in FIG. 7 indicate that the combined antibody treatment demonstrates an enhanced efficacy for killing B cells, especially at higher cell concentrations, where antibody and/or complement concentrations may limit efficacy. At lower plating densities, the combined antibody treatment appeared to provide about 34% killing, while the additive effect of each antibody tested separately at 215 ng/ml would be about 29% killing, thus demonstrating an effect that is at least additive, and possibly synergistic. At higher plating densities, the combined antibody treatment appeared to provide about 30% killing, while the additive effect of each antibody tested separately at 215 ng/ml would be about 23% killing, thus again demonstrating an effect that is at least additive, and possibly synergistic. The data shown is representative of one of three experiments.

### EXAMPLE 11

### Clinical Trial Treatment Protocol to Test the Efficacy of mAb 216 in Patients with ALL

This will be a phase I dose escalation study of human mAb 216 in children with relapsed or refractory B-precursor ALL. Two treatment courses of mAb infusion will be given, with the same dose of antibody administered on Day 0 and on Day 7.

Day 0: mAb 216 dose #1

Day 7: mAb 216 dose #2

Antibody Administration: Day 0 and Day 7

The mAb 216 should be diluted to a final volume of 1 mg/ml in normal saline at room temperature. The mAb solution should not be mixed or diluted with any other solutions or drugs. The initial dose rate at the time of the first mAb 216 infusion should be 25 mg/hour for the first half hour. If no toxicity or infusion-related event occurs, the dose rate may be escalated (25 mg/hour increments at 30 minute intervals) to a maximum of 200 mg/hour. Should any infusion-related toxicity occur, the antibody infusion should be temporarily slowed or interrupted, and the patient should be treated appropriately. When the symptoms improve, the infusion can be restarted at ½ the previous rate and gradually escalated to a maximum rate of 200 mg/hour.

Disease Evaluation and Pharmacokinetics

Early response to therapy will be performed on Day 7, prior to proceeding with second antibody infusion. Patients who have demonstrated a Good Response to the first dose of antibody will proceed to receive the second dose in identical fashion as on Day 0. Patients who have a Poor Response on Day 7 will receive the second dose of antibody in conjunction with vincristine. In the event that a patient clearly has a Poor Response to therapy by Day 5, i.e., has obvious rising peripheral blast count, the patient may proceed with the second dose of mAb216 with vincristine as early as Day 5.

Final response to antibody treatment will be performed on Day 35.

Pharmacokinetic sampling will be performed only with Dose # 1 of antibody infusion

mAb216 Dose Escalation Schedule

| Dose Levels | Dose (mg/kg) |
|---|---|
| Dose Level 1 | 1.25 |
| Dose Level 2 | 2.5 |
| Dose Level 3 | 5.0 |
| Dose Level 4 | 10.0 |
| Dose Level 5 | 20.0 |

The dose of mAb 216 will be calculated in mg per kg body weight as indicated above. Escalations are planned in groups of three patients, with an additional two patients to be added at the first indication of dose-limiting toxicity (DLT), as follows:

Three patients are treated at dose level one (1.25 mg/kg)

If none of the first three patients experiences a DLT, then the dose is escalated to the next level in three subsequent patients.

If one of three patients within a given cohort experiences a DLT, up to two additional patients will be treated at that level.

If neither of these two additional patients experience a DLT, then the dose is escalated for the subsequent cohort of patients.

If one or more of these two additional patients experience a DLT, patient entry at that dose level and further dose escalation will be stopped, and the MTD will have been exceeded. At least two additional patients will then be treated at the next lower dose level.

If two or more patients within any cohort (of three to five patients) experience a DLT, then the MTD will have been exceeded. At least two additional patients will then be treated at the next lower dose level.

The highest dose level reached at which no more than one of five patients experiences a DLT will be considered the MTD.

There will be no intra-patient dose escalation permitted on this study.

Definitions of Dose-Limiting Toxicity

Adverse events (toxicities) will be graded according to the NCI CTC v.2.0. DLT will be defined as any hematologic or nonhematologic toxicity that occurs that is at least (possibly, probably or definitely) attributable to the investigational agent, mAb 216.

Chemotherapy

Day 7 Evaluation: In the event that the Day 7 clinical response evaluation demonstrates POOR RESPONSE, defined as >25% leukemic blasts remaining on bone marrow examination (see section 5.0) or a rising peripheral blood blast count, Vincristine will be given on Day 7 PRIOR to initiating dose #2 of antibody. Vincristine will thereafter be administered weekly for 4 total doses according to the following schedule:

Vincristine 1.5 mg/m²/dose IVP on weekly×4 doses (Days 7, 14, 21, 28).

If a patient achieves a complete remission by Day 35 having received mAb 216+VCR on Day 7 followed by 3 additional weekly VCR doses, future treatment with mAb 216+VCR on a monthly basis may be possible, pending mAb 216 availability. The dose of mAb would remain the same as that given on Days 1 and 7 of protocol treatment.

Day 14, 21, and 28 Evaluations: In the event that the clinical response evaluation on Day 14, 21, or 28 reveals RESIDUAL DISEASE, defined as >5% leukemic blasts remaining on bone marrow examination, the patient may begin Reinduction Chemotherapy.

For patients who receive reinduction chemotherapy for Residual Disease on Day 14, 21, or 28, weekly BMA evaluations are no longer required for study purposes. It is recommended that patients who receive reinduction chemotherapy undergo BMA/LP approximately 4 weeks after initiating chemotherapy to assess remission status.

Reinduction Chemotherapy:

Reinduction chemotherapy is intended only for patients with residual disease detected at day 14, 21, or 28. A standard 4-drug, 28 day reinduction regimen includes:

Prednisone 40 mg/m²/day divided TID×28 days;

Vincristine 1.5mg/m²/dose IVP weekly×4 (Days 1, 8, 15, 22);

E. coli L-asparaginase 6,000 IU/m²/dose IM×6 doses

(Days 2, 5, 8, 12, 15, 19);

Daunomycin 30 mg/m²/dose IV weekly×3 doses

(Days 8, 15, 21);

Intrathecal methotrexate (age appropriate doses).

The treatment days begin with Day 1 as the first day of reinduction chemotherapy.

Days 1 and 15 (with additional doses days 8 and 22 if CNS 2, i.e., <5 WBC/μl and blasts on cytospin on Day 5 LP).

CNS Prophylaxis Dose:

| AGE | | MTX | VOLUME | |
|---|---|---|---|---|
| 1-1.99 | yr | 8 mg | 8 | cc |
| 2-2.99 | yr | 10 mg | 10 | cc |
| 3-3.99 | yr | 12 mg | 12 | cc |
| >9 | yr | 15 mg | 15 | c |

The above protocol allows the investigator to meet the following goals:

To estimate the maximum tolerable dose (MTD) of the VH4-34 encoded monoclonal antibody, mAb 216, administered in two doses one week apart, to children with relapsed or refractory acute lymphoblastic leukemia (ALL);

To determine the dose-limiting toxicities (DLT) of mAb 216 given on this schedule, as a single agent and in combination with vincristine;

To characterize the pharmacokinetic behavior of mAb 216 in children with relapsed or refractory ALL;

To preliminarily define the anti-tumor activity of mAb 216 within the confines of a phase I study; and

To assess the biologic activity of mAb 216 in patients with relapsed or refractory ALL.

### REFERENCES

1. Cook G P, Tomlinson I M, Walter G, Riethman H, Carter N P, Buluwela L, Winter G, Rabbitts T H. A map of the human immunoglobulin VH locus completed by analysis of the telomeric region of chromosome14q. Nat. Genet. 1994;7:162-168

2. Weng N P, Snyder J G, Yu L L, Marcus D M. Polymorphism of human immunoglobulin VH4 germ-line genes. Eur. J. Immunol. 1992;22:1075-1082

3. van der Maarel S, Willems van Dijk K, Alexander C, Sasso E, Bull A, Millner E. Chromosomal organization of the human VH4 gene family. J. Immunol. 1993;150:2858-2868

4. Pascual V, Victor K, Lesiz D, Spellerberg M B, Hamblin T J, Thompson K M, Randen I, Natvig J B, Capra J D, Stevenson F K. Nucleotide sequence analysis of the V regions of two IgM cold agglutinins. Evidence that VH4-21 gene segment is responsible for the major cross-reactive idiotype. J. Immunol. 1991;146:4385-4391

5. Pascual V, Victor K, Spellerberg M, Hamblin T J, Stevenson F K, Capra J D. VH restriction among human cold agglutinins. The VH4-21 gene segment is required to encode anti-I and anti-i specificities. J. Immunol. 1992;149:2337-2344

6. Silberstein L E, Jefferies L C, Goldman J, Friedman D, Moore J S, Nowell P C, Roelcke D, Pruzanski W, Roudier J, Silverman G J. Variable region gene analysis of pathologic human autoantibodies to the related i and I red blood cell antigens. Blood. 1991;78:2372-2386

7. Pruzanski W, Katz A. Cold agglutinins—antibodies with biological diversity. Clin. Immunol. Rev. 1984;3:131-168

8. Roelcke D. Cold agglutination. Transfusion Med. Rev. 1989;2:140-166

9. Stevenson F K, Smith G J, North J, Hamblin T J, Glennie M J. Identification of normal B-cell counterparts of neoplastic cells which secrete cold-agglutinins of anti-i and anti-I specificity. Br. J. Haematol. 1989;72:9-15

10. Kraj P, Friedman D F, Stevenson F, Silberstein L E. Evidence for the overexpression of the VH4-34 (VH4.21) Ig gene segment in the normal adult human peripheral blood B cell repertoire. J. Immunol. 1995;154:6406-6420

11. Pruzanski W, Roelcke D, Donnelly E, Lui L. Persistent cold agglutinins in AIDS and related disorders. Acta Haematologica. 1986;75:171-173

12. Ciaffoni S, Luzzati R, Roata C, Turrin I A, Antonello O, Aprili G. Presence and significance of cold agglutinins in patients with HIV infection. Haematologica. 1992;77:233-236

13. Isenberg D, Spellerberg M, Williams W, Griffiths M, Stevenson F. Identification of the 9G4 idiotope in systemic lupus erythematosus. Br J Rheumatology. 1993;32:876-882

14. Miller J J, Bieber M M, Levinson J E, Zhu S, Tsou E, Teng N N H. VH4-34 (VH4.21) Gene Expression in the Chronic Arthritides of Childhood: Studies of Associations with Anti Lipid A Antibodies, HLA, and Clinical Features. J. Rheumatol. 1996;23:2132-2139

15. van Vollenhoven R F, Bieber M M, Powell M J, Gupta P K, Bhat N M, Richards K L, Albano S A, Teng N N H. VH4-34 encoded antibodies in SLE: a specific diagnostic marker that correlates with clinical disease characteristics. J. Rheumatol., In press.

16. Chapman C J, Spellerberg M B, Smith G A, Carter S J, Hamblin T J, Stevenson F K. Auto anti-red cell antibodies synthesized by patients with infectious mononucleosis utilize the VH4-21 gene segment. J. Immunol. 1993;151:1051-1061

17. Grillot-Courvalin C, Brouet J-C, Piller F, Rassenti L Z, Labaume S, Silverman G J, Silberstein L, Kipps T J. An anti-B cell autoantibody from Wiskott-Aldrich syndrome which recognizes i blood group specificity on normal human B cells. Eur. J. Immunol. 1992;22:1781-1788

18. Bhat N M, Bieber M M, Chapman C J, Stevenson F K, Teng N N H. Human antilipid A monoclonal antibodies bind to human B cells and the ‘i’ antigen on cord red blood cells. J. Immunol. 1993; 151:5011-5021

19. Silberstein L E, George A, Durdik J M, Kipps T J. The V4-34 encoded anti-i autoantibodies recognize a large subset of human and mouse B cells. Blood Cells, Molecules, and Diseases. 1996;22:126-138

20. Bhat N M, Bieber M M, Hsu F J, Chapman C J, Spellerberg M, Stevenson F K, Teng N N H. Rapid cytotoxicity of human B lymphocytes induced by VH4-34 (VH4.21) gene encoded monoclonal antibodies, II. Clin. Exp. Immunol. 1997;108:151-159

21. Bhat N M, Bieber M M, Stevenson F K, Teng N N H. Rapid cytotoxicity of human B lymphocytes induced by VH4-34 (VH4.21) gene encoded monoclonal antibodies. Clin. Exp. Immunol. 1996;105:183-190

22. Bhat N M, Bieber M M, Young L W, Teng N N H. Susceptibility of B Cell Lymphoma to Human Antibodies Encoded by the V4-34 Gene. Critical Rev. Oncol/Hematol. 2001;39:59-68

23. Uckun F, Manivel C, Arthur D, Chelstrom L, al. e. In vivo efficacy of B43 (anti-CD19)-pokeweed antiviral protein immunotoxin against human pre-B cell acute lymphoblastic leukemia in mice with severe combined immunodeficiency. Blood. 1992;79:2201-2214

24. Khazaeli M B, Wheeler R, Rogers K, Teng N, Ziegler E, Haynes A, Saleh M N, Hardin J M, Bolmer S, Cornett J, Berger H, LoBuglio A F. Initial evaluation of a human immunoglobulin M monoclonal antibody (HA-1A) in humans. J. Biol. response. 1990;9:178-184

25. Fisher C J J, Zimmerman J, Khazaeli M B, al e. Initial evaluation of human monoclonal anti-lipid A antibody (HA-1A) in patients with sepsis syndrome. Crit Care Med. 1990;18:1311-1315

26. Ziegler E, Fisher C, Sprung C, Straube R, Sadoff J, Foulke G, Wortel C, Fink M, Dellinger P, Teng N, Allen E, Berger H, Knatterud G, LoBuglio A, Smith C. Treatment of gram-negative bacteremia and septic shock with HA-1A human monoclonal antibody against endotoxin. N. Engl. J. Med. 1991;324:429-436

27. Hardin J, Khazaeli M, Allen I, C D, LoBuglio A,. Limited sampling models for HA-1A IgM monoclonal antibody. Hum Antibodies Hybridomas. 1990:115-119

28. McCloskey R V, Straube R C, Sanders C, Smith S M, Smith C R. Treatment of septic shock with human monoclonal antibody HA-1A. A randomized, double-blind, placebo-controlled trial. CHESS trial study group. Ann. Intern. Med. 1994; 121:1-5

29. Derkx B, Wittes J, McCloskey R. Randomized, placebo-controlled trial of HA-1A, a human monoclonal antibody to endotoxin, in children with meningococcal septic shock. European Pediatric Meningococcal Septic Shock Trial Study Group. Clin Infect Dis. 1999;28:770-777

30. Romano M J, Kearns G L, Kaplan S L, Jacobs R F, Killian A, Bradley J S, Moss M M, Van Dyke R, Rodriguez W, Straube R C. Single-dose pharmacokinetics and safety of HA-1A, a human IgM anti-lipid-A monoclonal antibody, in pediatric patients with sepsis syndrome. J Pediatr. 1993;122:974-981.

1. A method of treating a human patient suffering from a condition characterized by a hyperproliferation of B cells, comprising contacting said B cells with (1) a cytotoxic amount of an antibody having specific binding for CDIM epitopes on a B cell, and (2) a cytotoxic agent.

2. The method of claim 1, wherein the condition characterized by a hyperproliferation of B cells is lymphoid cancer, viral infection, immunodeficiency, or autoimmune disease.

3. The method of claim 2, wherein said viral infection is human immunodeficiency virus or mononucleosis.

4. The method of claim 2, wherein said immunodeficiency is post-transplant lymphoproliferative disease, or immunodeficiency syndrome

5. The method of claim 2, wherein said autoimmune disease is systemic lupus erythematosis, rheumatoid arthritis, autoimmune lymphoproliferative disease, multiple sclerosis, psoriasis, myasthenia gravis, Hashimoto's thyroiditis, Alzheimer's disease, lupus nephritis, Class III autoimmune diseases such as immune-mediated thrombocytopenias, such as acute idiopathic thrombocytopenic purpura and chronic idiopathic thrombocytopenic purpura, dermatomyositis, Sjogren's syndrome, multiple sclerosis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, Crohn's disease, sarcoidosis, ulcerative colitis, erythema multiforme, IgA nephropathy, polyarteritis nodosa, ankylosing spondylitis, Goodpasture's syndrome, thromboangitis ubiterans, primary biliary cirrhosis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephritis and fibrosing alveolitis,.

6. The method of claim 1, wherein the cytotoxic agent is a chemotherapeutic agent, a radioactive isotope, a cytotoxic antibody, an immunoconjugate, a ligand conjugate, an immunosuppressant, a cell growth regulator and/or inhibitor, a toxin, or mixtures thereof.

7. The method of claim 6, wherein the chemotherapeutic agent is an agent that disrupts the cytoskeleton of the B cell.

8. The method of claim 7, wherein the agent that disrupts the cytoskeleton of the B cell is an agent that interferes with the polymerization or depolymerization of microtubules.

9. The method of claim 8, wherein the agent that interferes with the polymerization or depolymerization of microtubules is a taxane, vinca alkaloid or colchicine, or mixtures thereof.

10. The method of claim 9, wherein the vinca alkaloid is vinblastine, vincristine, vindesine, or vinorelbine, or mixtures thereof.

11. The method of claim 9, wherein the taxane is paclitaxel, or docetaxel, or mixtures thereof.

12. The method of claim 8, wherein the agent that disrupts the cytoskeleton of the B cell is an anti-actin agent.

13. The method of claim 6, wherein the chemotherapeutic agent is asparaginase, epipodophyllotoxin, camptothecin, antibiotic, platinum coordination complex, alkylating agent, folic acid analog, pyrimidine analog, purine analog or topoisomerase inhibitor, or mixtures thereof.

14. The method of claim 13, wherein the topoisomerase inhibitor is an epipodophyllotoxin.

15. The method of claim 13, wherein the epipodophyllotoxin is etoposide or teniposide.

16. The method of claim 13, wherein the pyrimidine analog is capecitabine, 5-fluoruracil, 5-fluorodeoxyuridine, 5-fluorodeoxyuridine monophosphate, cytosine arabinoside, 5-azacytidine, 2′, 2′-difluorodeoxycytidine.

17. The method of claim 13, wherein the purine analog is mercaptopurine, azathioprene, thioguanine, pentostatin, erythrohydroxynonyladenine, cladribine, vidarabine, fludarabine phosphate.

18. The method of claim 13, wherein the folic acid analog is methotrexate, raltitrexed, lometrexol, permefrexed, edatrexate, pemetrexed.

19. The method of claim 13, wherein the camptothecin is irinotocan, topotecan, camptothecan.

20. The method of claim 13, wherein the antibiotic is dactinomycin, daunorubicin, doxorubicin, idarubicin, epirubicin, valrubucin, mitoxanthrone, bleomycin, or mitomycin.

21. The method of claim 13, wherein the platinum coordination complex is cisplatin, carboplatin, or oxaliplatin.

22. The method of claim 13, wherein the alkylating agent is mechlorethamine, cyclophosphamide, ifosfamide, melphalan, dacarbazine, temozolomide, thiotepa, hexamethylmelamine, streptozocin, carmustine, busulfan, altretamine or chlorambucil.

23. The method of claim 1, wherein the cytotoxic agent is administered simultaneously with, before or after administration of the antibody having specific binding for CDIM epitopes on a B cell.

24. The method of claim 1, wherein the cytotoxic agent is covalently or noncovalently attached to the antibody having specific binding for CDIM epitopes on a B cell.

25. The method of claim 1, wherein the antibody having specific binding for CDIM epitopes on a B cell is a VH4-34 encoded antibody.

26. The method of claim 25, wherein the antibody having specific binding for CDIM epitopes on a B cell is mAb 216, RT-2B, FS 12, A6(H4C5), Cal-4G, S20A2, FS 3, Gee, HT, Z2D2, Y2K.

27. The method of claim 26, wherein the antibody having specific binding for CDIM epitopes on a B cell is mAB 216.

28. The method of claim 1, wherein the antibody having specific binding for CDIM epitopes on a B cell comprises a CDR sequence having a net positive charge.

29. The method of claim 6, wherein the cytotoxic agent is a radioactive isotope.

30. The method of claim 29, wherein the radioactive isotope is ¹³¹I, ¹²⁵I, ¹²³I, ⁹⁰Y, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁸Re or ¹⁸⁶Re.

31. The method of claim 6, wherein the cytotoxic antibody has specific binding for a cell surface receptor on a B cell.

32. The method of claim 31, wherein the cytotoxic antibody has specific binding for CD11a, CD19, CD20, CD21, CD22, CD25, CD34, CD37, CD38, CD40, CD45, CD52, CD80, CD 86, IL4R, IL-6R, IL-8R, IL-13, IL-13R, α-4/β-1 integrin (VLA4), BLYS receptor, cell surface idiotypic Ig, tumor necrosis factor (TNF), or mixtures thereof.

33. The method of claim 31, wherein the cytotoxic antibody is efalizumab (RAPTIVA), rituximab (RITUXAN), daclizumab (ZENAPAX), epratuzumab, basiliximab (SIMULECT), anti-CD52 (CAMPATH), natalizumab, or infliximab (REMICADE).

34. The method of claim 31, wherein the cytotoxic antibody is an immunoconjugate.

35. The method of claim 6, wherein the cytotoxic agent is a ligand conjugate.

36. The method of claim 35, wherein the ligand conjugate comprises IL-2, IL-4, IL-6, IL-13, IL-15, BLYS, or TNF.

37. The method of claim 6, wherein the immunosuppressant is a glucocorticoid, a calcineurin inhibitor, an antiproliferative/antimetabolic agent, or an immunosuppressive antibody.

38. The method of claim 37, wherein the calcineurin inhibitor is cyclosporine or tacrolimus.

39. The method of claim 37, wherein the antiproliferative/antimetabolic agent is azathioprine, chlorambucol, cyclophosphamide, leflunomide, mycophenolate mofetil, methotrexate, rapamycin, thalidomide, or mixtures thereof.

40. The method of claim 37, wherein the glucocorticoid is selected from prednisolone, prednisone, or dexamethasone.

41. The method of claim 37, wherein the cell growth regulator and/or inhibitor is a small molecule therapeutic agent, gene therapy agent or gene expression modifier.

42. The method of claim 41, wherein the small molecule therapeutic agent is a kinase inhibitor, or a proteasome inhibitor.

43. The method of claim 42, wherein the kinase inhibitor is a bcr/abl tyrosine kinase inhibitor, or a tyrosine kinase inhibitor.

44. The method of claim 42, wherein the proteasome inhibitor is a boronic ester.

45. The method of claim 41, wherein the gene therapy agent is a plasmid, naked DNA, or nucleic acid-peptide complex.

46. The method of claim 41, wherein the gene expression modifier is an antisense nucleic acid, or interfering nucleic acid (e.g., RNAi).

47. The method of claim 6, wherein the toxin is Pseudomonas exotoxin A, ricin, diphtheria toxin, momordin, pokeweed antiviral protein, Staphylococcal enterotoxin A, gelonin, or a maytansinoid.

48. The method of claim 2, wherein said lymphoid cancer is any acute or chronic leukemia or lymphoma of B cell origin.

49. The method of claim 2, wherein the lymphoid cancer is acute lymphocytic leukemia (ALL), non-Hodgkins lymphoma (NHL), Burkitt's lymphoma, B progenitor ALL, adult ALL, or chronic lymphocytic leukemia (CLL).

50. The method of claim 1, wherein said contacting is performed in vivo, in vitro or ex vivo.

51. The method of claim 50, wherein said in vivo contacting is performed by administering said antibody having specific binding for CDIM epitopes on a B cell by parenteral injection to the human patient.

52. A method of treating a human patient suffering from a condition characterized by lymphoid cancer, comprising administering (1) a cytotoxic amount of an antibody having specific binding for CDIM epitopes on a B cell, and (2) a chemotherapeutic agent.

53. A method of treating a human patient suffering from a condition characterized by lymphoid cancer, comprising administering (1) a cytotoxic amount of an antibody having specific binding for CDIM epitopes on a B cell, and (2) a cytotoxic antibody having specific binding for a cell surface receptor on a B cell.

54. A method of augmenting the B cell cytotoxicity of an antibody that binds a CDIM epitope, comprising contacting B cells with the antibody that binds a CDIM epitope and an agent that disrupts the cytoskeleton of B cells.

55. The method of claim 54, wherein the agent that disrupts the cytoskeleton of B cells is an agent that interferes with the polymerization or depolymerization of microtubules.

56. The method of claim 55, wherein the agent that interferes with the polymerization or depolymerization of microtubules is a taxane, vinca alkaloid or colchicine.

57. The method of claim 56, wherein the vinca alkaloid is vinblastine, vincristine, vindesine, or vinorelbine.

58. The method of claim 56, wherein the taxane is paclitaxel, or docetaxel.

59. The method of claim 54, wherein the method of augmenting B cell cytotoxicity is used in the therapy of lymphoid cancer, B cell hyperproliferative diseases, or autoimmune disease.

60. A method of treating an autoimmune disease in a mammal, comprising administering (1) a cytotoxic amount of an antibody having specific binding for CDIM epitopes on a B cell, and (2) a chemotherapeutic agent, an antibody having specific binding for cell surface receptors on a B cell, an immunosuppressant, a cell growth regulator and/or inhibitor, or mixtures thereof.

61. A method of killing malignant B cells that are resistant to chemotherapeutic agents, cell growth regulators and/or inhibitors, or cytotoxic antibodies, comprising contacting said malignant B cells with an antibody having specific binding for CDIM epitopes on a B cell.

62. The method of claim 61, further comprising contacting said malignant B cells with an additional chemotherapeutic agent.

63. A method of killing malignant B cells that are resistant to an antibody having specific binding for CDIM epitopes on a B cell, comprising treating said B cells with a chemotherapeutic agent and an antibody having specific binding for CDIM epitopes on the B cells.

64. A method of treating a disease or disorder characterized by a hyperproliferation of B cells, comprising contacting the B cells with an amount of an antibody having specific binding for CDIM epitopes on a B cell sufficient to permeabilize the B cells.

65. A method for reducing tumor load in a patient suffering from lymphoid cancer refractory to reinduction therapy, comprising administering a cytotoxic amount of an antibody having specific binding for CDIM epitopes on a B cell, wherein said treatment allows the patient to undergo subsequent reinduction therapy.

66. A method for augmenting the cytotoxicity of an anti-B cell antibody, comprising administering a cytotoxic amount of an antibody having specific binding for CDIM epitopes on a B cell.

67. A pharmaceutical formulation for parenteral injection comprising a cytotoxic amount of an antibody having specific binding for CDIM epitopes on a B cell.

68. The pharmaceutical formulation of claim 67, further comprising a chemotherapeutic agent.

69. A kit for treating a patient suffering from a condition characterized by a hyperproliferation of B cells comprising:
(a) an amount of an antibody having specific binding for CDIM epitopes on a B cell sufficient to permeabilize the B cells in the patient,
(b) a therapeutically effective amount of a cytotoxic agent effective to treat the condition characterized by the hyperproliferation of B cells.

70. A method for purging the bone marrow of a patient suffering from lymphoid cancer of malignant B cells prior to reimplantation of the bone marrow in the patient after myeloablative therapy, comprising treating the bone marrow ex vivo with a cytotoxic amount of an antibody having specific binding for CDIM epitopes on a B cell.

71. The method of claim 70, further comprising treating the bone marrow with a cytotoxic agent.

72. A method for treating a human patient suffering from a condition characterized by hyperproliferation of B cells, comprising contacting said B cells with (1) a cytotoxic amount of the anti-CDIM antibody mAb 216 or Y2K, and (2) an anti-CD20 antibody.

73. The method of claim 72, wherein the anti-CD20 antibody is rituximab, tosutimab or ibritumomab.

74. The method of claim 72, further comprising contacting the B cells of the patient with vincristine or vinblastine.

75. A method for treating a human patient suffering from a condition characterized by hyperproliferation of B cells, comprising contacting said B cells with (1) a cytotoxic amount of the anti-CDIM antibody mAb 216 or Y2K, and (2) an anti-CD52 antibody.

76. The method of claim 75, wherein the anti-CD52 antibody is CAMPATH.

77. The method of claim 75, further comprising contacting the B cells of the patient with vincristine or vinblastine.

78. A method for treating a human patient suffering from a condition characterized by hyperproliferation of B cells, comprising contacting said B cells with (1) a cytotoxic amount of the anti-CDIM antibody mAb 216 or Y2K, and (2) an anti-CD22 antibody.

79. The method of claim 78, wherein the anti-CD22 antibody is epratuzumab.

80. The method of claim 78, further comprising contacting the B cells of the patient with vincristine or vinblastine.
